(19) United States
(12) Patent Application Publication
(10) Pub. No.: US 2003/0219878 A1
(43) Pub. Date: Nov. 27, 2003

(54) Sticky rice

(75) Inventors: John A. Lindbo, Vacaville, CA (US); Hal S. Padgett, Vacaville, CA (US)

Correspondence Address: LARGESCALE BIOLOGY CORPORATION, BANK OF AMERICA PLAZA, SUITE 2020, 414 UNION STREET, NASHVILLE, TN, 37219, US

(73) Assignee: LARGE SCALE BIOLOGY CORPORATION, Vacaville, CA

(21) Appl. No.: 10/286,549

(22) Filed: Nov. 1, 2002

Related U.S. Application Data
(63) Continuation-in-part of application No. 10/190,451, filed on Jul. 2, 2002.
(60) Provisional application No. 60/365,058, filed on Mar. 13, 2002.

(51) Int. Cl. C12Q 1/68; C12P 19/34

(52) U.S. Cl. 435/91.2; 435/6

(57) ABSTRACT

Restriction Independent Cloning Events (RICE) are made by generating 5′ overhangs (sticky ends). The polynucleotides to be joined are reacted with a DNA polymerase, having 3′ to 5′ exonuclease activity and 5′ to 3′ polymerizing activity, less than all of the dNTPs, a kinase (optional) and a ligase. The complementary 5′ overhangs anneal and ligate.

### RELATED APPLICATIONS

The instant application claims priority to co-pending U.S. Non-provisional patent application Ser. No. 10/190,451, filed Jul. 2, 2002, entitled STICKY RICE, which claims priority to U.S. provisional patent application No. 60/365,058 filed Mar. 13, 2002, entitled STICKY RICE, both of which are incorporated herein by reference in their entirety.

### BACKGROUND OF THE INVENTION

The present invention relates to a method of joining polynucleotides by reacting polynucleotides with a DNA polymerase, a DNA ligase and optionally a polynucleotide kinase. Complementary 5′ cohesive (sticky) ends are generated and subsequently anneal and are ligated all in the same reaction vessel. The method allows for the formation of Restriction Independent Cloning Events (RICE) or Restriction Independent Cohesive Ends (RICE) by reacting sticky ended polynucleotides, i.e., Sticky RICE.

U.S. Pat. No. 5,075,227 discloses methods for cloning cDNA and producing mRNA from the cloned c-DNA. The c-DNA is cloned in one orientation by using a vector containing a directional cloning site and primer-adapter sequences complementary to portions of the directional cloning site. U.S. Pat. No. 5,075,227 describes directional sticky end cloning where sticky end (5′ overhangs) are generated by treatment of linear double stranded (ds) DNAs with T4 DNA polymerase and only one dNTP. Sticky ends of 12-15 nucleotides are disclosed as being preferred in U.S. Pat. No. 5,075,227. However, the vector polynucleotides and insert polynucleotides in the U.S. Pat. No. 5,075,227 are reacted with DNA polymerase separately and cannot be co-treated and the T4 DNA polymerase activity must be inactivated before the vector and insert polynucleotides are combined.

The present invention provides an easy and quick method for joining multiple polynucleotides in a single tube or vessel and a mechanism for directional cloning without the requirement for restriction enzymes and minimal primer modification.

### SUMMARY OF THE INVENTION

Briefly, in accordance with the present invention two or more double stranded polynucleotides are joined by generating complementary 5′ cohesive ends on two or more polynucleotides by reacting the polynucleotides with a DNA polymerase having 3′ to 5′ exonuclease activity and 5′ to 3′ polymerizing activity in the presence of less than all of the dNTPs selected from the group consisting of dGTP, dCTP, dATP and dTTP wherein complementary 5′ overhangs (cohesive ends) are produced. The complementary cohesive ends are allowed to anneal. The annealed polynucleotides are then ligated in the presence of DNA ligase. If the polynucleotides do not have a 5′ phosphate group, a 5′ phosphate group may be formed in situ by adding a polynucleotide kinase to the reaction mixture.

The present process generates the complementary sticky ends using the 3′ to 5′ exonuclease activity of the DNA polymerase and allows the complementary sticky ends to anneal. The annealed complementary sticky ends are then ligated with a DNA ligase. These steps can all be done concurrently by incubating all of the components of the reactions together. Alternatively, the present process can be conducted in a sequential manner by first reacting the polynucleotides with the DNA polymerase, appropriate dNTP(s) and optional kinase before the DNA ligase and/or kinase is added to the reaction mixture.

Of particular interest in practicing the present inventions, vector (double stranded polynucleotides) fragments and an insert double stranded DNA fragment polynucleotide are reacted with a T4 DNA polymerase, appropriate dNTP(s), a kinase and T4 DNA ligase. Complementary 5′ overhangs are generated, and are allowed to anneal and ligate resulting in insertion of the DNA insert into the vector. The finished vector is then used to transform a host.

### BRIEF DESCRIPTION OF THE DRAWINGS

FIG. 1 is a photograph of a gel that has been digitally inverted such that all dark areas are light and all light areas are dark to reveal the details of the banding displayed in the gel more clearly, and shows the results of experiments as described below in Example 1A, 1B, 1C and 1D, where lane 1 in FIG. 1 is Promega 100 bp DNA molecular weight ladder, lane 2 shows Example 1A reaction, lane 3 shows Example 1B reaction, lane 4 shows Example 1C reaction and lane 5 shows Example ID reaction.

FIG. 2 is a diagram that represents Experiment 2A, 2B, 2C and 2D and shows plasmid pUC 19 and relative locations and directions of the primers used for amplification of pUC 19 by PCR for experiments in Examples 2A-2D. In the diagram, primers are represented by the solid arrows, 5′ prime (blunt end of arrow) to-3′ prime (point of arrow). Primers Seq ID NO: 18, Seq ID NO: 22, Seq ID NO: 26, and Seq ID NO: 30 hybridize to pUC 19 at the location identified by primer B in FIG. 2. Primers Seq ID NO: 19, Seq ID NO: 23, Seq ID NO: 27, and Seq ID NO: 31 hybridize to pUC 19 at the location identified by primer A in FIG. 2.

FIG. 3 is a photograph of a gel that has been digitally inverted such that all dark areas are light and all light areas are dark to reveal the details of the banding displayed in the gel more clearly, and indicates the results of experiments as described below in Example 2 showing Sticky RICE cloning of inserts into pUC using T4 DNA ligase.

FIG. 4 is a photograph of a gel that has been digitally inverted such that all dark areas are light and all light areas are dark to reveal the details of the banding displayed in the gel more clearly. The gel indicates the results of experiments as described below with respect to Example 3 showing Sticky RICE cloning of inserts into pUC using E. coli ligase.

FIG. 5 is a photograph of a gel that has been digitally inverted such that all dark areas are light and all light areas are dark to reveal the details of the banding displayed in the gel more clearly. FIG. 5 indicates the experimental results corresponding to Example 4 showing Sticky RICE cloning of GFP PCR product into SapI digested vector DNA—pLSB 1176.

FIG. 6 is a photograph of a gel that has been digitally inverted such that all dark areas are light and all light areas are dark to reveal the details of the banding displayed in the gel more clearly. The results indicated in FIG. 6 are explained in greater detail below in Example 5.

FIG. 7 is a photograph of a gel that has been digitally inverted such that all dark areas are light and all light areas are dark to reveal the details of the banding displayed in the gel more clearly. The results indicated in FIG. 7 are explained in greater detail below in Example 6.

FIG. 8A is a photograph of a gel that has been digitally inverted such that all dark areas are light and all light areas are dark to reveal the details of the banding displayed in the gel more clearly. FIG. 8 indicates results of directional joining of a 225 and 725 bp PCR product sharing a 3 bp overlap as described below in Example 7.

FIG. 8B is a flowchart showing the basic steps described in Example 7.

FIG. 9A is a photograph of a gel that has been digitally inverted such that all dark areas are light and all light areas are dark to reveal the details of the banding displayed in the gel more clearly. FIG. 9A indicates the results of Example 8, described in greater detail below.

FIG. 9B is a flowchart showing the basic steps described in Example 8.

FIG. 10A is a flowchart showing other embodiments of the methods for joining DNA fragments in accordance with the present invention.

FIG. 10B is a flowchart showing yet another embodiment of the methods for joining DNA fragments in accordance with the present invention.

FIG. 10C is a flowchart showing still another embodiment of the methods for joining DNA fragments in accordance with the present invention.

FIG. 10D is a flowchart showing still yet another embodiment of the methods for joining DNA fragments in accordance with the present invention.

FIG. 11 is a flowchart showing yet another embodiment of the present invention.

FIG. 12 is a flowchart showing yet another embodiment of the present invention.

FIG. 13 is a chart showing a partial sequence listing for pLSB 1176 that was assembled using the methods of the present invention, with key portions marked for discussion below.

FIG. 14 is a flowchart showing basic steps for converting PCR products into expressible elements without cloning in accordance with further embodiments of the present invention.

FIG. 15 is a flowchart that depicts a variety of combinations of steps for practicing the present invention in a two step process, the flowchart corresponding to Examples 9-16 of the present invention.

FIGS. 16A, 16B and 16C are flowcharts that depict a further variety of combinations of steps for practicing the present invention in a three step process, the flowcharts corresponding to Examples 17-33 of the present invention.

FIG. 17 is a flow chart that depicts a still more combinations of steps for practicing the present invention, the flowchart corresponding to Examples 34-41 of the present invention.

FIG. 18 is a block diagram depicting a computer and fluid handler for effecting the various procedures described herein in an automated fashion.

### DETAILED DESCRIPTION OF THE INVENTION

In practicing the present invention, double stranded polynucleotides are joined by generating 5′ overhangs (cohesive or sticky ends) of defined length and sequence by employing a DNA polymerase having 3′ to 5′ exonuclease activity. This is accomplished by reacting the DNA polymerase with a linearized polynucleotide (ds DNA molecule) and a subset of at least one type of dNTP but not more than three of the four dNTPs (dATP, dTTP/dUTP, dCTP or dGTP). The dsDNA molecule can be blunt or non-blunt ended. After the 5′ overhang cohesive ends are generated the complementary cohesive ends are allowed to anneal whereby at least one of the ds DNA molecules has a 5′ phosphate group. The 5′ phosphate group can be present on the ds DNA molecule before the reaction or can be formed in situ during the reaction by addition of a polynucleotide kinase to the reaction mixture. After annealing, the cohesive ends are ligated in the presence of a DNA ligase.

The polynucleotides or ds DNA molecules to be joined according to the present invention can be any ds polynucleotides where it would be desirable to join them to make a longer polynucleotide molecule. The polynucleotides can be prepared using conventional techniques such as known DNA synthesis techniques, DNA polymerase chain reaction (PCR) or by restriction enzyme digestion, such as, for example, with a type IIS restriction enzyme, such as Sap I or Ear I. When a restriction endonuclease is used to make the starting polynucleotide the resulting polynucleotide can be blunt ended or can have either a 3′ or 5′ overhang. In a preferred embodiment, the polynucleotides to be joined comprise a vector and an insert polynucleotide wherein the insert includes the coding region for the expression of a desirable protein or RNA molecule. Once the vector and insert polynucleotides are joined forming a finished vector, the finished vector is used to transform a host cell whereby the host contains the desirable coding region and is capable of expressing the encoded protein or RNA molecule. The joined polynucleotide can be either linear or circular. The present process can be used to clone a single gene of interest or to clone a library of polynucleotides into a suitable vector. Another preferred embodiment includes cloning of DNA sequences which may or may not encode for protein or RNAs.

The 5′ overhangs, also referred to as sticky ends or cohesive ends, can be 1-15 nucleotides in length and are preferably 2-8 nucleotides in length. The 5′ overhangs are comprised of any of the nucleotides but can be comprised of the same nucleotide, i.e., all t's, all g's, etc.). When the '5 overhang is comprised of mixed nucleotides it is preferred that the 5‘overhang is comprised of a’s and t's together or g's and c's together. The complementary 5′ overhangs allow the polynucleotides to be joined in a directional orientation if desired. The 5′ overhang can also be self-complementary to an otherwise identical polynucleotide.

The following 5′ overhangs and their complementary sequence are useful in practicing the present invention (however, other 5′ overhangs can also be used):

| | |
|---|---|
| 5′ Overhang | Complementary Sequence |

| | |
|---|---|
| 5′ ttt ttt | aaa aaa 5′ |
| 5′ gg gg | cc cc 5′ |
| 5′ cc | gg 5′ |
| 5′ aaaat | tttta 5′ |
| 5′ ggc | ccg 5′ |
| 5′ atg | tac 5′ |
| 5′ aaa | ttt 5′ |
| 5′ taa | att 5′ |
| 5′ cgg | gcc 5′ |
| 5′ gcc | cgg 5′ |
| 5′ aat | tta 5′ |

The length of the 5′ cohesive/sticky ends formed by the DNA polymerase activity is determined by the presence of one or more stop bases that occur consecutively in the polynucleotide sequence. The stop bases, in conjunction with the appropriate dNTPs, limit the 3′ to 5′ exonuclease activity of the DNA polymerase enzyme thereby limiting the length of the 5′ single stranded ends that are formed. The overhang sequences and/or stop bases are conveniently incorporated into the primers that are used in the PCR process when PCR is employed to make the starting polynucleotides. The overhang and stop bases can also overlap between the primer and original nucleotide. Alternatively, the stop bases and 5′ overhang are already in the polynucleotide when the polynucleotides are obtained without PCR.

The following reaction scheme illustrates the role of stop bases and dNTPs in the present invention. Fragment C and Fragment D represented below are to be joined according to the Sticky RICE process. The stop bases in Fragment C are “GG” and the stop bases in Fragment D are “CC.”

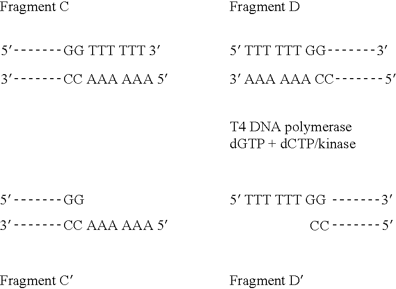

The dNTPs employed in the Sticky RICE process for joining the above DNA fragments are dGTP and dCTP. The dGTP limits the exonuclease activity of T4 DNA polymerase on Fragment C resulting in Fragment C′ having the desired 5′ overhang. The dCTP limits the exonuclease activity of the T4 DNA polymerase on Fragment D resulting in Fragment D′ having a desired 5′ overhang. The 5‘overhangs of Fragments C’ and Fragment D′ are complementary.

The DNA polymerase, kinase and ligase enzymes used in the present process are those enzymes that are well known to one of ordinary skill in the art. The DNA polymerase will have 5′ to 3′ polymerase activity and also 3′ to 5′ exonuclease activity. Suitable DNA polymerases include but are not limited to T4 DNA polymerase, E. coli DNA polymerase 1, the Klenow fragment of E.-coli DNA polymerase and T7 DNA polymerase. The ligase is capable of creating a phosphodiester between the 3′ hydroxyl of one nucleotide and the 5′ phosphate of another nucleotide. Suitable ligases include, but are not limited to, T4 DNA ligase and E. coli DNA ligase. The kinase is capable of adding a phosphate group to the 5′ end of a polynucleotide. Suitable kinases include, but are not limited to, T4 polynucleotide kinase. The kinase is an optional component of the present method and is employed to place a phosphate group on the 5′ end of the polynucleotides if the polynucleotides do not already contain a 5′ phosphate group. The kinase conveniently adds the phosphate group in situ in the reaction mixture.

In one embodiment of the present invention the DNA polymerase, polynucleotide kinase and DNA ligase reactions are accomplished concurrently by incubating or reacting all of the components together for a time and under conditions sufficient to allow the generation of the complementary 5′ overhangs (cohesive ends), annealing of the complementary 5′ overhangs and their resulting ligation. Alternatively, the present process can be conducted sequentially where the DNA polymerase and/or kinase reaction is allowed to proceed to substantial completion before the DNA ligase and/or kinase reaction. The kinase reaction, if needed to add 5′ phosphate groups to the polynucleotides, is conducted either before the DNA ligase reaction or concurrently therewith. Alternatively the DNA polymerase and/or kinase reaction can be performed and then the DNA polymerase and/or kinase activities can be inactivated (by a high temperature incubation step, for example 75 C for 10 minutes) which is known to inactivate the DNA polymerase and/or kinase enzymes used. Following this inactivation, ligase and/or kinase activities can be added to the reaction.

The present process or method is conducted employing standard reactants, procedures and equipment well known to one of ordinary skill art. Buffers when employed are also well known to the skilled artisan. The temperature at which the present reactions are conducted is not critical. Temperatures are advantageously from about 5° C. to about 50° C. and preferably from about 12° C. to about 37° C. The temperature of the different reactions can vary (DNA polymerase reaction conducted at a different temperature than the ligation reaction, for example) or they can be conducted at substantially the same temperature. One skilled in the art can easily determine the optimum temperature based on the particular reactants and reagents by conducting routine experiments at various temperatures.

Since enzymes are involved, the length of the reaction period will vary inversely with the temperature, i.e., in general the higher the temperature the quicker the reaction.

In one embodiment of the present invention a vector polynucleotide and an insert polynucleotide are reacted in the presence of a subset of dNTPs, T4 DNA polymerase, a kinase and T4 DNA ligase. The reaction mixture is incubated for 30 minutes at 37° C. and then allowed to further incubate at room temperature overnight. An alternative incubation period would be to allow the reactants to incubate at between 16° C. and ambient temperature overnight. Alternatively, the vector polynucleotide and the insert polynucleotide can be reacted with the subset of d NTPs and T4 DNA polymerase at 37° C. for 30 minutes followed by the addition of T4 DNA ligase and kinase after the reaction mixture has cooled to room temperature. This final reaction mixture is then allowed to incubate at room temperature for 12-16 hours or overnight. It is readily apparent that the present process allows the simultaneous treatment of both the vector polynucleotide and the insert polynucleotide with the DNA polymerase and dNTPs wherein the complementary 5′ overhangs are produced in situ. Additionally, the DNA polymerase does not need to be inactivated before the ligase reaction is allowed to proceed, but can be if so desired.

The following reaction scheme illustrates “Sticky RICE” joining of two ds polynucleotides where ds polynucleotide Fragment A and ds polynucleotides Fragment B are joined:

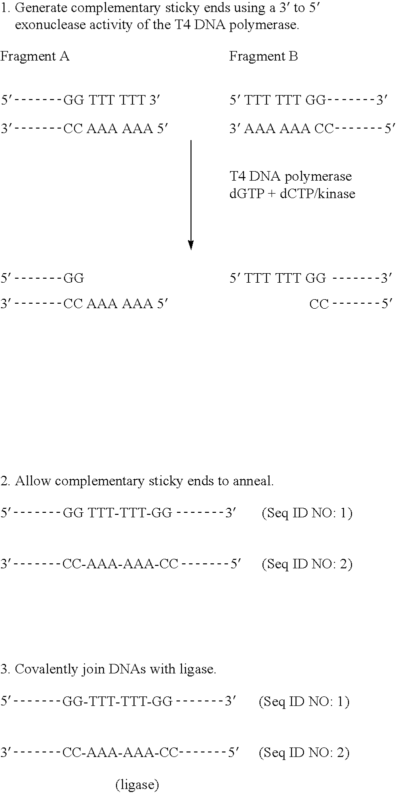

The method of the present invention is useful in constructing linear expression elements, in gene/vector assembly, for constructing gene fusions, for ligating into plasmids or phage or for any other procedures where a recombinant polynucleotide is the desired product. Single gene cloning or library construction can also be accomplished. The present method is adaptable, easy to use, cost effective from materials, time and labor perspectives and, efficient. The method allows directional cloning and is seamless, i.e., no restriction sites are required.

In another embodiment of the present sticky RICE reaction, it may be desirable to purify the joined DNA reaction product from the buffers and/or enzymes used in the sticky RICE reaction. This can be accomplished by placing a suitable tag moiety (such as a biotin group, for example) on at least one of the starting material DNAs to be used in the sticky RICE reaction. The incorporation of biotin tags into synthetic DNAs is well known to the skilled artisan. Oligos containing one or more biotin groups are commercially available. Such biotinylated oligos can be used in polymerase chain reactions. After the “tagged” reaction products are formed then standard purification protocols relating to the “tagged” products are employed. For example, a ds DNA product comprising an open reading frame of interest is synthesized with a biotinylated “downstream” or reverse oligonucleotide is joined with a second ds DNA product which contains additional vector components, for example, a phage RNA polymerase promoter sequence (such as the T7, SP6 or T3 RNA polymerase promoters) according to the present Sticky RICE process resulting in the ligation of the two DNA fragments. The biotinylated DNA product is readily purified from the sticky RICE reaction components by a variety of known reagents, such as magnetic beads coated with avidin or streptavidin molecules. After collecting the magnetic beads to which the biotinylated DNAs are now bound, the beads are washed and ultimately transferred into a separate reaction, such as an in vitro transcription reaction, for example. Streptavidin coated magnetic beads and protocols for their use in purification of biotinylated DNAs are available from a number of commercial suppliers, including the Promega Corporation (Madison, Wis., USA), and Dynal (Lake Success, NY, USA).

The present invention can be employed to generate directional cloning kits using a desired vector. These kits would facilitate the use of the vectors for whatever their intended purpose (for example library construction, cloning genes for expression of proteins in animal, plant or bacterial cells, etc.) by making the cloning process easier and more efficient. A cloning kit would include:

a. a DNA polymerase

b. a polynucleotide kinase

c. a ligase and

d. a buffer, and optionally the following:

e. a linearized cloning vector

f. competent cells

g. dNTPs

h. PCR buffer

i. Taq DNA polymerase or other thermostable DNA polymerase

One or more control reactions may also be included in the cloning kit. The first three listed enzymes could be combined into a mixture.

A typical cloning kit is as follows:

Sticky RICE Enzyme mix (1 U T4 DNA polymerase; 1 U T4 Polynucleotide kinase; 0.25 U T4 DNA ligase)

Sticky RICE buffer mix

Linearized cloning vector

Competent Cells

Control primers

Control template

dNTPS

PCR Buffer

Taq DNA polymerase

2 control reactions.

The present invention also allows for the generation of a fully automated or nearly fully automated cloning system. By using robotic liquid handlers, etc. an automated workflow for cloning PCR products into a plasmid vector capable of replicating in a bacterial cell (for example) could be set up:

Step 1—set up PCR reactions to amplify up polynucleotide sequences of interest.

Step 2—analyze PCR reactions on gel, destroy template (if desired), purify PCR product away from unincorporated dNTPs. Estimate DNA concentration of purified PCR products.

Step 3—set up Sticky RICE cloning reactions by combining Sticky RICE compatible vector with specific amounts of various PCR products.

Step 4. let Sticky RICE reaction proceed for appropriate length of time.

Step 5. transform all or a portion of Sticky RICE reaction into bacterial cells.

Step 6 plate bacteria, pick colonies, prep up DNA from colonies, or cultures thereof, continue with recombinant DNA in experiments as desired.

In another embodiment the generation of linear expression elements for gene discovery/functional genomics experiments, two hybrid interaction experiments, etc. without cloning genes (without propagating the recombinant DNA in cells, for example) can be accomplished with the present process. This work flow would be as follows and could be automated as well:

Step 1—set up PCR reactions to amplify up polynucleotide sequences of interest.

Step 2—analyze PCR reactions on gel, destroy template (if desired), purify PCR product away from unincorporated dNTPs. Estimate DNA concentration of purified PCR product.

Step 3—set up Sticky Rice cloning reactions by combining Sticky RICE compatible polynucleotide elements to 5′ and/or 3′ ends of PCR products. These “elements” (described in Step 3) would generally be PCR products of polynucleotide sequences with specific useful features such as: promoters, 5′ untranslated leader sequences, poly adenylation signals, transcription terminators, stop codons, or open reading frames (ORFs) that could be ligated to a PCR product in frame with the ORF of the PCR product—this would allow the production of fusion proteins at either N or C terminus. Desirable proteins for this application would include Green fluorescent protein (GFP), polyhistidine (His) tag sequences, cellulose binding domains, streptavidin binding peptide sequences, epitope tags, etc. These fusions would be useful for detection of the recombinant protein, and/or aid in its purification and/or detection, etc.

Step 4: after attaching “elements” of interest to 5′ and/or 3′ end of your PCR product, an additional PCR step (to amplify up more of the newly constructed recombinant DNA) may be desired.

Step 5. clean up DNA as needed.

Step 6: use linear expression element in either in vitro or in vivo settings for transcription and/or translation of DNA of interest. For example, linear expression elements can be transfected into mammalian cells and be expressed transiently. Additionally, PCR products could be transcribed and translated in vitro using commercially available systems (Promega Corp, Ambion Corp, etc.).

The advantages of the present Sticky RICE process are flexibility and speed. In hours one can PCR up more of a DNA of interest for immediate use. In contrast, it takes days to transfect DNA into bacteria, let the bacteria grow, then purify recombinant DNA from bacterial culture. Also the original PCR product can be modified in different ways as desired, providing flexibility in expressing native or fusion proteins in vivo or in vitro.

The following is a first embodiment of a kit with instructions for using STICKY RICE™:

STICKY RICE™ DNA Cloning kit.

For Directional Cloning of PCR products into Plasmids

Kit Contents and Materials List

Introduction

Overview

Experimental Outline

Methods

Primer Design for STICKY RICE

PCR Amplification of DNAs

Post-PCR Clean up of DNAs

STICKY RICE™ Ligation Reaction Protocol

Transformation of competent E. coli cells

Analysis of transformants

Troubleshooting and Control Reactions

Technical Assistance

Contact information

Appendix

Kit Contents and Materials List

Kit Contents

The STICKY RICE™ Cloning kit contains the following reagents:

| Item | Contents | Amount |
|---|---|---|
| Control Insert DNA | 3 ng/ul 725 bp DNA | 25 ul |
| STICKY RICE ™ Prepared | ˜10 ng/ul | 40 ul |
| Vector (pLSB 1200.bsr) | | |
| Sterile Water | dH₂O | 500 ul |
| 5X STICKY RICE ™ Buffer | 5X buffer | 50 ul |
| STICKY RICE ™ | 10Xenzyme mix | 30 ul |
| Enzyme Mix | | |
| Control PCR Template | 10 ng/ul | 20 ul |
| Control Primer set 1 | 50 uM (each) | 20 ul |
| | Forward and Reverse | |
| | primer mix. | |

Store all kit components at −20° C.

Note: Control insert is GFPc3 ORF and will be cloned in frame with the N-terminal His-Patch thioredoxin ORF and C-terminal V5, 6×His ORF in pLSB1200.bsr.

Materials Supplied by the User

Thermocycler (PCR) machine.

Gene Specific Primers

PCR reagents

PCR Clean up kit (Stratagene, Qiagen, etc.)

Competent Bacteria Cells (we recommend MaxEfficiency chemically competent DH5α)

Introduction

Overview:

This kit is designed to let you rapidly clone PCR products in a directional orientation into the bacterial expression vector pLSB 1200.bsr without the use of restriction enzymes. The vector is provided is ready to use, along with all other necessary reagents for the cloning reaction. The kit contains all the components needed to perform 10 independent cloning reactions. The Instant RICE Enzyme Mix provided allows for cloning of DNAs at room temperature (22° C.) in 30 minutes. Following the STICKY RICE reaction the DNA is transformed into chemically competent E. coli cells. Approximately 95% of the transformants should contain inserts in the proper orientation.

The prepared pLSB 1200.bsr vector provided is a bacterial expression vector with the following features:

i T7 lac promoter for high level-inducible expression of the gene of interest.

ii. LacI gene encoding the lac repressor to tightly regulate gene expression.

iii. Ampicillin resistance for selection in E. coli

iv. pBR322 origin for low-copy replication and maintenance in E. coli.

v. Directional STICKY RICE™ compatable cloning sites.

vi. N-terminal His-Patch thioredoxin fusion protein production.

vii. C-terminal V5 6X His fusion, if desired.

To clone a gene of interest into pLSB 1200.bsr, the gene is simply amplified up by the polymerase chain reaction (PCR) and the PCR reaction product purified away from unincorporated nucleotides and primers. The cleaned up PCR product is then combined with STICKY RICE vector, buffer and enzyme mix.

If the PCR product is inserted in the proper orientation in pLSB 1200.bsr, a StuI site will be generated at the upstream vector:insert junction and a HindIII site will be generated at the 3′ Insert:vector junction. This provides an efficient way to screen for the presence and size of an insert

Introduction

Methods

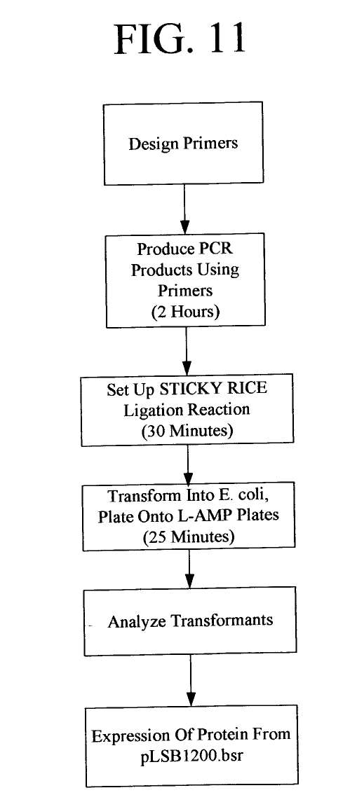

For efficient, directional joining of PCR products minor primer design constraints are necessary. The basic steps of the present invention described below are depicted in FIG. 11 and described below:

1. Design Primers:

### Forward Primer Design

The forward primer of your gene of interest must have the following 5′ terminal sequence:

5′ ggC CTT X₁X₂X₃ XXX XXX

Where X₁X₂X₃ is the first codon of your gene of interest. These 6 nucleotides (5′ggCCTT) ensure that your gene of interest is cloned in frame with the upstream Thioredoxin gene.

### Reverse Primer Design

The reverse primer of your gene of interest must have the following 5′ terminal sequence (of 4 nts):

| | |
|---|---|
| Gene Sequence: | XXX XXX X₁X₁₁Xᵢᵢᵢ |
| Primer Sequence | X′X′X′X′X′X′X′X′X′TTC g 5′ |

Where XᵢXᵢᵢXᵢᵢᵢ is a codon of the gene of interest and X′ is a nucleotide base complementary to X. NOTE: if XᵢXᵢᵢXᵢᵢᵢ is NOT a stop codon, then the gene will be cloned in frame with the downstream V5-6×His orf, resulting in a C-terminal V5-6XHis fusion to your protein.

2. PCR Amplification of DNAs using properly designed primers

Set up a 100 ul PCR reaction using the following guidelines:

Follow manufacturer's recommendations.

Use the cycling parameters suitable for your primers and template.

Use a 7 minute final extension time to ensure that all PCR products are completely extended.

After cycling, place the tube on ice or at −20C and proceed to Checking the PCR product, below.

Checking the PCR Product

Remove 5 ul from each PCR reaction and use agarose gel electrophoresis to verify the quality and quantity of your PCR product. Check for the following:

Be sure you have a single, discrete band of the correct size. If you do not have a single, discrete band, follow the manufacturer's recommendations for optimizing your PCR with the polymerase of your choice. Alternatively, you may gel-purify the desired product using the kit or method of your choice. We have successfully used Stratagene but have not tested other manufacturers.

Proceed to Post-PCR Clean up step.

Post-PCR Clean-Up of DNAs

a. Prior to cleaning up your PCR product, it is recommended you destroy the PCR template. To do this add 20 units DpnI restriction enzyme to your 100 ul PCR reaction. (DpnI is active in PCR buffer.) Digest template for 1 hour at 37° C. Then proceed to step b, below.

b. Purify your PCR product away from unincorporated dNTPs and primers using a DNA clean up kit from the manufacturer of your choice.

Suitable kit manufacturers include: Stratagene PCR clean up kit, Qiaquick PCR clean up kit. (We have not yet tried other manufacturers kits).

Follow manufacturers instructions and elute final DNA product in sterile dH₂O or 10 mM Tris pH 8.0.

Estimation of DNA Concentration

Estimation of the cleaned up PCR product DNA concentration can be done by agarose gel electrophoresis or UV absorption analysis.

Dilute (or concentrate) your cleaned up PCR product as needed. The recommended amounts of PCR product per STICKY RICE Cloning reaction is 0.015 pmoles. See Step 3, below for more details.

3. STICKY RICE™ Ligation Reaction Protocol

Assemble the STICKY RICE™ Ligation Reaction as follows:

2 ul 5× Reaction buffer

2 ul STICKY RICE™ Vector DNA (110 ng/ul)

y ul PCR product (0.015 pmoles insert DNA 2)

1 ul STICKY RICE™ Enzyme MIX (add last)

volume to 10 ul with sterile water.

Mix gently by pipetting up and down.

Incubate reaction for 30 minutes at room temperature.

Note: Each 10 ul STICKY RICE™ Ligation reaction contains ˜20 ng, or 0.005?pmoles, of prepared vector. It is recommended you use 0.015 pmoles of insert per reaction. Use the table below to determine the amount of DNA (in nanograms) you need to add depending upon the size of the DNA fragment to be cloned. (In general multiply the insert size (in kb) by 10 to determine the number of nanograms needed per 10 ul STICKY RICE™ Ligation reaction.

| PCR Product Size | 0.015 pmoles |
|---|---|
| 0.1 kb | 1 ng |
| 0.5 kb | 5 ng |
| 0.75 kb | 7.5 ng |
| 1 kb | 10 ng |
| 1.5 kb | 15 ng |
| 2 kb | 20 ng |
| 2.5 kb | 25 ng |
| 3 kb | 30 ng |
| 3.5 kb | 35 ng |
| 4 kb | 40 ng |
| 4.5 kb | 45 ng |
| 5 kb | 50 ng |
| 5.5 kb | 55 ng |
| 6 kb | 60 ng |
| 6.5 kb | 65 ng |

4. Transformation of Competent E. coli Cells.

Thaw competent DH5a chemically competent cells on ice.

We have used Invitrogen Max Efficiency DH5α, E. coli (>1×10⁹ transformation efficiency).

Place 14 ml polypropylene tube Falcon (35/2059) on ice to pre-chill.

Transfer 35 ul thawed cells to chilled 14 ml polypropylene tube

Add 1 ul STICKY RICE™ Ligation reaction to competent DH5a cells in 14 ml tube Swirl gently to mix.

Incubate on ice 15 min.

Transfer to 42° C. water bath for 45 seconds.

Return to ice 2 minutes.

Add 400 ul 50C media (room temp) to cells.

Plate 50 or 150 ul per plate onto LB Amp plates (100 ug/ml Ampicillin)

Incubate plates at 37° C. overnight.

5. Analysis of Transformants.

Using a toothpick, pick and inoculate 2 ml LB (100 ug/ml ampicillin) samples with individual colonies. Pick at least 6 independent colonies.

Grow liquid cultures overnight at 37° C. and shaking (300 rpm)

Prepare DNA from liquid cultures using Qiagen plasmid DNA miniprep kit.

Screen plasmids for inserts by digestion with StuI and HindIII.

We recommend digesting 2 ul of miniprep DNA in a 15 ul reaction containing 0.5 ul (each) of StuI and HindIII restriction enzymes. Digest for 1 hour at 37C.

Run restriction enzyme digested samples on 1% agarose gel to analyze.

After the StuI-HindIII digest, the pLSB 1200.bsr vector will be approximately 6.3 kb in length. Any additional fragments will be due to your insert.

6. Expression of Protein in E. coli from pLSB1200.bsr Vector.

Transform plasmid DNA from isolates of interest into E. coli BL21 DE3 cells (Invitrogen) as per manufacturers instructions. To produce protein in E. coli follow instructions for induction and expression of protein(s).

Troubleshooting and Control Reactions:

The STICKY RICE™ reaction components and protocol are designed to maximize specific, directional joining of PCR products, while minimizing the amount of non-specific, non-directional joining of PCR products. Several elements contribute to this specificity, including:

1. DNA concentration.

High concentrations of DNA facilitate blunt DNA ligation reactions. Increasing the DNA concentration above the amounts recommended in this manual may lead to non-specific (blunt) joining of PCR products.

2. Enzyme concentration.

The STICKY RICE™ Enzyme Mix is specially formulated to limit the amount of non-specific joining of PCR fragments.

Appendix:

Appendix Table of Contents:

Control Primer Sequences:

Reagents and Supplies list

STICKY RICE™ Buffer recipe

STICKY RICE™ Enzyme mix recipe

Preparation of Vector

Preparation of Control Insert

Control STICKY RICE™ Ligation reaction and Results

Plasmid map of pLSB 1200.bsr

pLSB 1200.bsr cloning sites

Control Primer Sequences

Forward primer Seq ID NO: 3: ggC CTT ATg gCT AgC AAA ggA gAA gAA C (IDT Inc. Coralville, Iowa)

Reverse primer Seq ID NO: 4: gCT TgT AgA gCT CAT CCA TgC CAT g (IDT Inc. Coralville, Iowa)

Control PCR template: GFPc3 ORF in pUC-based cloning vector.

Reagents and Supplies List:

DNA purification Reagents:

Successful results have been obtained with the following kits.

QIAGEN plasmid DNA prep system

Stratagene plasmid DNA prep system

Stratagene PCR clean up kit.

Zymoclean Gel Purification kit.

Restriction Enzymes

New England Biolabs (NEB) Sap I (Cat # R0569) 2 U/ul

NEB SnaB I (Cat # R0130) 5 U/ul

NEB Hind III (Cat # R0104) 20U/ul

NEB Stu I (Cat # R0187) 10U/ul

NEB Dpn I (Cat # R0176) 20U/ul

DNA Modification Enzymes:

T4 DNA polymerase. Novagen LIC qualified (Cat #70099-3) 2.5U/ul

T4 polynucleotide kinase: NEB (Cat #M0201) 10 U/ul

T4 DNA ligase (Invitrogen Cat # 15224-017) 1U/ul

dNTPs

Promega (Cat #U1330) set of 4 individual dNTPs each at 100 mM

Buffers:

NEB Restriction enzyme buffers 2 and 4

NEB T4 DNA ligase buffer

Composition of 10×buffer:

500 mM Tris-HCl (pH 7.5)

100 mM MgCl₂

100 mM DTT

10 mM rATP

250 ug/ml BSA

PCR Reagents:

Pfu Turbo DNA polymerase. (Stratagene Cat #600250) 2.5 U/ul

10× cloned Pfu buffer (Stratagene)

STICKY RICE™ Buffer Mix:

50 ul 2 mM (each) dATP/dTTP

50 ul NEB 10×T4 DNA ligase buffer.

Result: 100 ul of 5×STICKY RICE™ Buffer

STICKY RICE™ Enzyme Mix:

10 ul Novagen T4 DNA pol (2.5U/ul)

5 ul NEB T4 polynucleotide kinase (10U/ul)

50 ul Gibco (Invitrogen) T4 DNA ligase (1U/ul)

Result: 65 ul of 10× STICKY RICE™ enzyme mix.

Preparation of pLSB 1200.bsr Vector for STICKY RICE™ Cloning.

NOTE: This is HOW the supplied vector has been prepared. A user of the present invention does not need to prepare the supplied vector.

A. Purify pLSB 1200.bsr vector from DH5a E. coli cultures using Stratagene or QIAGEN plasmid DNA purification systems.

B. Digest pLSB 1200.bsr vector DNA as follows:

10 ul 10×NEB 4 buffer

1 ug pLSB 1200.bsr DNA

1 ul 100×BSA (NEB)

5 ul Sap I Restriction enzyme

Volume to 100 ul with dH₂O.

Digest at 37° C. for about 16 hours (ex, 5 pm to 8 am)

Add 1 ul SapI (2U/ul stock) and 1 ul SnaBI (5 U/ul stock) to 100 ul digest

Return to 37° C. 1 hour.

C. Purify digested DNA with Stratagene or QIAGEN PCR clean up systems as per manufacturers instruction with the following modification: Perform one extra column wash than suggested by protocol.

Elute in 50 ul dH20.

D. Run 2 ul of purified DNA on 1% agarose gel to estimate quality and/or quantity of DNA.

Alternatively, record A260/A280 readings of DNA (100 ul of a 1:20 dilution) to estimate DNA concentration.

Adjust cut vector DNA concentration to about 5 ng/ul in dH₂O.

Store at −20° C.

Preparation of Control Insert:

NOTE: This is HOW the supplied control insert has been prepared. A user of the present invention does not need to prepare control insert, unless he so desires.

Set up a PCR of the (ca. 725 bp) GFP control insert as follows:

10 ul 10× cloned Pfu buffer (Stratagene)

50 pmoles F primer (SEQ ID NO: 3)*

50 pmoles R primer (SEQ ID NO: 4)

10 ng template (GFP gene in pUC based plasmid)

2 ul dNTP mix (dATP, dTTP, dGTP, dCTP) each at 10 mM

2 ul Pfu Turbo DNA Polymerase

Volume to 100 ul with dH₂O

50 pmoles=1 ul of 50 uM stock.

Cycling conditions (MJ Research PTC 200)

1. 95° C. 2 min

2. 94° C. 30 sec

3. 55° C. 30 sec (steps 2-4 for 25 cycles)

4. 72° C. 1 min

5. 72° C. 7 min

6. 4° C. hold

Add 20 U Dpn I to PCR reaction when completed.

Incubate at 37° C. 1 hour (to destroy template DNA)

Run 2 ul of PCR reaction on 1.5% agarose gel to check.

Clean up PCR product with Stratagene or QIAGEN PCR clean up kits, according to manufacturers instructions.

Elute in 50 ul dH₂O

Run 2 ul cleaned up PCR product on gel to check.

Record A260/A280 of cleaned up PCR product (1:50 dil) to estimate DNA concentration.

Adjust concentration of cleaned up PCR product to approximately 3-4 ng/ul, for use as test insert.

Control STICKY RICE ™
Ligation reaction and Results

| Reagent | Control Lig | Test Lig |
|---|---|---|
| 5X Buffer | 2 ul | 2 ul |
| Vector | 10 ng | 10 ng |
| Control Insert | — | 7.5 ng |
| Water | up to 9 ul vol | up to 9 ul vol |
| STICKY RICE ™ | 1 ul | 1 ul |
| Enzyme Mix | | |
| Final vol | 10 ul | 10 ul |

Mix gently by pipetting.

Let incubate at room temp for 30 min.

Transform 1 ul each reaction into 35 ul DH5a, plate cells etc. as described in protocol.

Incubate overnight at 37° C.

Record the number of colonies per plate next day.

NOTE: A decent vector prep should give about 10×-40× more colonies on the ligation reaction than on the re-ligation control reaction. Re-ligation control should have 20-50 colonies, for example per 50 ul plated

Expected results: >or =to 95% of transformants contain plasmids with inserts.

The following is a second embodiment of a kit with instructions for using STICKY RICE™:

STICKY RICE™ Linear DNA Recombination Kit.

For Construction of linear recombinant DNAs in vitro.

Kit Contents and Materials List

Introduction

Overview

Experimental Outline

Methods

Primer Design for STICKY RICE

PCR Amplification of DNAs

Post-PCR Clean up of DNAs

STICKY RICE™ Reaction Protocol

Gel Analysis of Reaction products

Troubleshooting and Control Reactions

Technical Assistance

PCR Control Reaction

STICKY RICE Control Reaction

Appendix

Recipes

Technical Service

References

Kit Contents and Materials List

Kit Contents The STICKY RICE™ Linear Recombination kit contains the following reagents:

| Item | Composition | Amount |
|---|---|---|
| Control DNA-1 | 8 ng/ul 225 bp frag | 25 ul |
| Control DNA-2 | 25 ng/ul 725 bp frag | 25 ul |
| Control DNA-3 | 10 ng/ul 300 bp frag | 25 ul |
| Sterile Water | | 500 ul |
| 5X STICKY RICE ™ | 5X buffer | 50 ul |
| Buffer | | |
| STICKY RICE ™ | 10Xenzyme mix | 30 ul |
| Enzyme Mix | | |
| Control Template | 10 ng/ul | 20 ul |
| Control Primer Set 1 | 50 uM Each primer | 20 ul |
| Control Primer Set 2 | 50 uM Each primer | 20 ul |
| Control Primer Set 3 | 50 uM Each primer | 20 ul |
| Primer Seq ID NO: 6 (F) | 50 uM Seq ID NO: 6 primer | 20 ul |
| Primer Seq ID NO: 13 (R) | 50 uM Seq ID NO: 13 primer | 20 ul |

Materials Supplied by the User

Thermocycler (PCR) machine

PCR Reagents

PCR Clean up kit

Introduction

### Overview

This kit is designed to let you rapidly join linear DNA (PCR) products in a directional orientation. The STICKY RICE™ Enzyme Mix facilitates joining of linear DNAs at room temperature (22° C.) in 60 minutes. Greater than 50% of the control reaction substrates should be directionally joined in a 60 minute STICKY RICE™ joining reaction.

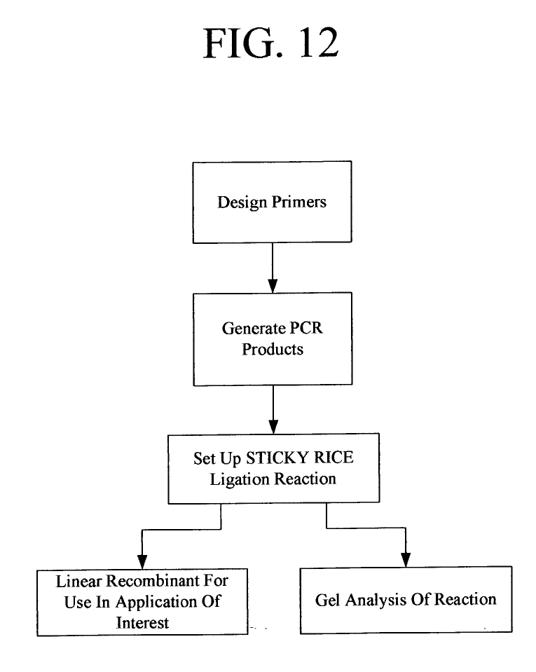

For efficient, directional joining of PCR products minor primer design constraints are necessary (for complete description see Methods section). The provided control PCR reaction products of 225 (DNA-1) and 725 bp (DNA-2) for example, are joined (at their overlapping GCC sequence termini) to generate a product of about 950 bp. See FIG. 12. Alternatively template and primers are provided to allow you to amplify up these two PCR products for use in control reactions. Instructions for primer design will allow you to generate your own PCR products for use in a STICKY RICE™ joining procedure. You may also set up a 3 way STICKY RICE™ reaction by adding in equimolar amounts of control DNA-3. The basic steps of the method described here are depicted in FIG. 12.

FIG. 12 depicts the joining of 225 and 725 bp PCR products by STICKY RICE™.

```
^{5′} ---225----GCC3′ (DNA-1 in kit)
_{3′}----225----CGG5′
                              ^{5′}GCC----725bp -------^{3′} (DNA-2 in kit)
                              _{3′}CGG----725bp--------_{5′}
5′ -------225bp------GCC--------725 bp-----3′
3′-------225bp------CGG--------725 bp-----5′
```

The STICKY RICE™ joining procedure is useful for joining PCR products for the construction of chimeric genes, gene fusions, or other linear recombinant DNAs of interest.

Introduction

SEE FIG. 12 for Experimental Steps

Methods

Design Primers for STICKY RICE™

During the STICKY RICE™ Ligation reaction, 5′ complementary (sticky) overhangs are generated and guide the directional assembly of DNA molecules. The efficiency and rate of the joining reaction correlates with the calculated melting temperature of the generated 5′ overhangs. The higher the estimated melting temperature of the sticky ends, the faster the reaction progress.

For this reason, G/C rich sticky ends are preferred over A/T rich sticky ends, though both types of sticky ends work. The sequence and length of the 5′ sticky ends to be generated is determined by primer design prior to PCR. For example 1f two DNA fragments (left) and (right) are to be joined the following design is suggested. Left Fragment, Reverse primer (5′ GGC W-gene of interest sequence)

### EXAMPLE

| | |
|---|---|
| DNA Sequence: | NNN NNN |
| Primer Sequence: | NNN NNN W CGG5′ |

where W=A or T

The forward direction primer for the Left fragment is a gene specific primer.

Right fragment, forward primer (5′ GCC W-gene of interest sequence)

### EXAMPLE

| | | | |
|---|---|---|---|
| DNA Sequence: | gene of interest-- | ATG GGC ATT | (Seq ID NO:5) |
| Primer Sequence: | | 5′ GCC ATG GGC ATT | |

Reverse primer for the Right fragment is a gene specific primer.

PCR Amplification of DNAs

Set up a 100 μl PCR reaction using the following guidelines:

Follow manufacturer's recommendations.

Use the cycling parameters suitable for your primers and template.

Use a 7 minute final extension time to ensure that all PCR products are completely extended.

It is recommended to set up the control fragments in the following manner:

| | |
|---|---|
| Pfu turbo(2.5 U/ul) | 2 μl |
| 10X cloned Pfu buffer | 10 μl |
| 10 mM dNTPs | 2 μl |
| Control primers set 1 or 2 | 1 μl |
| control template | 2 μl |
| sterile water | 83 μl |

Using an MJ research thermocycler (use of a different PCR machine will require optimization of the cycling conditions), the reaction is heated to 95° C. for 2 minutes then cycled for 25 times through the following three temperatures: 95° C., 30 seconds; 55° C., 30 seconds; 72° C. 1 minute. After the final cycle, the reaction is incubated at 72° C. for 7 minutes and then held constant at 4° C. until ready for next step.

After cycling, place the tube on ice or at −20C and proceed to Checking the PCR product, below.

Checking the PCR Product

Remove 2-5 ul from each PCR reaction and use agarose gel electrophoresis to verify the quality and quantity of your PCR product. Check for the following:

Be sure you have a single, discrete band of the correct size. If you do not have a single, discrete band, follow the manufacturer's recommendations for optimizing your PCR with the polymerase of your choice. Alternatively, you may gel-purify the desired product using the method of your choice. We have used Stratagene based kits

Proceed to Post-PCR Clean up step.

Post-PCR Clean-Up of DNAs

Before cleaning up PCR fragments, add 20 units DpnI per 100 ul reaction to remove template background. Incubate DpnI treated sample for 1 hour at 37 degrees celcius. Heat kill enzyme for 20 minutes at 80 degrees celcius. Proceed with purification of PCR products.

Purify your PCR product away from unincorporated dNTPs and primers using a DNA clean up kit from the manufacturer of your choice.

Suitable kit manufacturers include: Stratagene PCR clean up kit, Qiaquick PCR clean up kit.

Follow manufacturers instructions and elute final DNA product in sterile dH₂O

Estimation of DNA Concentration

Estimation of the cleaned up PCR product DNA concentration can be done by agarose gel electrophoresis or UV absorption analysis.

Dilute (or concentrate) your cleaned up PCR product as needed. The recommended amounts of PCR products per STICKY RICE™ joining reaction are approximately 0.05 pmoles each fragment per 15 ul reaction (for 2-way ligation reactions). When joining two PCR products equimolar amounts of each PCR product are recommended. For 3-way ligations, it is recommended that the middle fragment be in excess of the other two fragments. A 1:3:1 molar ratio is recommended-0.025 pmoles of each outside fragment and 0.075 pmols of the inside fragment.

Control PCR products of 225 bp, 725 bp and 300 bp are supplied with the kit at 8 ng, 25 ng, and 10 ng/ul respecitively. One microliter of each fragment is 0.05 pmoles.

3. Set up STICKY RICE™ Ligation Reaction.

Assemble the STICKY RICE™ Reaction as follows:

3 ul 5× Reaction buffer

x ul DNA 1 (0.05 pmoles DNA 1 or 1 μl of control DNA-1)

y ul DNA 2 (0.05 pmoles DNA 2 or 1 μl of control DNA-2)

1 ul STICKY RICE™ Enzyme Mix (added last) volume to 15 ul with sterile water.

Mix by pipetting up and down gently or vortexing very gently

Incubate reaction for 60 minutes at room temperature (22° C.).

NOTE: Alternatively, if you wish to try a 3-way STICKY RICE™ Ligation Reaction, add 0.025 pmoles (0.5 μl of DNA-1 and DNA-3) of the 225 bp and 300 bp (provided) and 0.075 pmoles of (1.5 ul of DNA-2) to the above recipe and make adjustments accordingly to the amount of water added to the reaction. In the 3 way STICKY RICE™ Ligation the final product will be approximately 1300 bp in length. The fragments should assemble in the following orientation:

DNA1 (225 bp): DNA2 (725 bp): DNA3 (300 bp)

TABLE 1
Amount of DNA (in nanograms)
0.05 pmoles

| PCR Product Size | 0.05 pmoles |
|---|---|
| 0.1 kb | 3.3 ng |
| 0.5 kb | 16 ng |
| 0.75 kb | 25 ng |
| 1 kb | 33 ng |
| 1.5 kb | 50 ng |
| 2 kb | 66 ng |
| 2.5 kb | 83 ng |
| 3 kb | 99 ng |

4. Gel Analysis of Reaction

Stop Reaction:

Incubate the STICKY RICE™ Ligation Reaction at 75° C. for 15 minutes. This step is important to ensure you will have sharp bands on your gel.

Use agarose gel electrophoresis to examine the progress of each STICKY RICE™ Reaction. Run 10 ul of STICKY RICE Ligation Reaction on the appropriate concentration agarose gel, with the appropriate DNA size markers. Stain with EtBr and visualize fragments with UV illumination.

For the control reaction provided with this kit, a 1.5% agarose gel is recommended for the analysis of the control reaction. The user should see a 225 and 725 bp band in addition to a 950 bp band from the joining of control DNAs 1 and 2.

5. Linear Recombinant for use in your Application of Interest.

If desired, you may wish to PCR amplify up the assembled 1.3 kb recombinant TM DNA from the 3 way STICKY RICE™. To do this, use oligos Seq ID NO: 15 (225 F) and Seq ID NO: 13 and 1 ul of your STICKY RICE™ ligation reaction in the following PCR set-up

| | |
|---|---|
| Pfu turbo (2.5 U/ul) | 2 μl |
| 10X cloned Pfu buffer | 10 μl |
| 10 mM dNTPs | 2 μl |
| 50 μM Seq ID NO: 15 | 1 μl |
| 50 μM Seq ID NO: 13 | 1 μl |
| ligation product | 1 μl |
| sterile water | 83 μl |

Using an MJ research thermocycler (use of a different PCR machine will require optimization of the cycling conditions), the reaction is heated to 95° C. for 2 minutes then cycled for 20-25 times through the following three temperatures: 95° C., 30 seconds; 50° C., 30 seconds; 72° C. 1 minute, 30 seconds. After the final cycle, the reaction is incubated at 72° C. for 7 minutes and then held constant at 4° C. until ready for next step. Check 2 ul on a 1.2-1.5% agarose gel. You should only see the ligation product band as a result of PCR.

Troubleshooting and Control Reactions:

The STICKY RICE™ reaction components and protocol are designed to maximize specific, directional joining of PCR products, while minimizing the amount of non-specific, non-directional joining of PCR products. Several elements contribute to this specificity, including:

1. DNA concentration.

High concentrations of DNA facilitate blunt DNA ligation reactions.

Increasing the DNA concentration above the amounts recommended in this manual may lead to non-specific (blunt) joining of PCR products.

2. Enzyme concentration.

The Instant RICE™ Enzyme Mix is specially formulated to limit the amount of non-specific joining of PCR fragments.

3. Primer design.

Palidromic sticky ends can lead to non-directional annealing and joining of DNA fragments. To ensure that joining is directional design your primers so that non-palindromic sticky 5′ overhangs will result.

Appendix:

Primer pairs for 3 way STICKY RICE™ PCR products:

| | |
|---|---|
| 225 bp PCR product: (from pUC:GFP template) | |
| F primer (Seq ID NO: 6) 5′ gac tct tca agc gca aga gcg ccc aat acg ca 3′ | |
| R primer (Seq ID NO: 7) 5′ ggc tta gct gtt tcc tgt gtg aaa ttg tt 3′ | |
| 225 bp fragment sequence: | |
| GACTCTTCAAGCGCAAGAGCGCCCAATACGCAAACCGCCTCTCCCCGCGCGTTGGCCGATTC | (Seq ID NO: 8) |
| ATTAATGCAGCTGGCACGACAGGTTTCCCGACTGGAAAGCGGGCAGTGAGCGCAACGCAATT | |
| AATGTGAGTTAGCTCACTCATTAGGCACCCCAGGCTTTACACTTTATGCTTCCGGCTCGTAT | |
| GTTGTGTGGAATTGTGAGCGGATAACAATTTCACACAGGAAACAGCTAAGCC | |
| 725 bp PCR product (from pUC:GFP template) | |
| F primer (Seq ID NO: 9) 5′ gcc atg gct agc aaa gga gaa gaa c 3′ | |
| R primer (Seq ID NO: 10) 5′ cgg tta ttt gta gag ctc atc cat gcc a 3′ | |
| 726 bp fragment sequence: | |
| GCCATGGCTAGCAAAGGAGAAGAACTTTTCACTGGAGTTGTCCCAATTCTTGTTGAATTAGA | (Seq ID N): 11) |
| TGGTGATGTTAATGGGCACAAATTTTCTGTCAGTGGAGAGGGTGAAGGTGATGCTACATACG | |
| GAAAGCTTACCCTTAAATTTATTTGCACTACTGGAAAACTACCTGTTCCATGGCCAACACTT | |
| GTCACTACTTTCTCTTATGGTGTTCAATGCTTTTCCCGTTATCCGGATCATATGAAACGGCA | |
| TGACTTTTTCAAGAGTGCCATGCCCGAAGGTTATGTACAGGAACGCACTATATCTTTCAAAG | |
| ATGACGGGAACTACAAGACGCGTGCTGAAGTCAAGTTTGAAGGTGATACCCTTGTTAATCGT | |
| ATCGAGTTAAAAGGTATTGATTTTAAAGAAGATGGAAACATTCTCGGACACAAACTCGAGTA | |
| CAACTATAACTCACACAATGTATACATCACGGCAGACAAACAAAAGAATGGAATCAAAGCTA | |
| ACTTCAAAATTCGCCACAACATTGAAGATGGATCCGTTCAACTAGCAGACCATTATCAACAA | |
| AATACTCCAATTGGCGATGGCCCTGTCCTTTTACCAGACAACCATTACCTGTCGACACAATC | |
| TGCCCTTTCGAAAGATCCCAACGAAAAGCGTGACCACATGGGCCTTCTTGAGTTTGTAACTG | |
| CTGCTGGGATTACACATGGCATGGATGAGCTCTACAAATAACCG | |
| 300 bp PCR product (from pUC:GFP template DNA) | |
| F primer (Seq ID NO: 12) 5′ ccg ttt taa gcc agc ccc gac cac ccg cca 3′ | |
| R primer (Seq ID NO: 13) 5′ gat gag cgg ata cat att tga atg 3′ | |
| 300 bp fragment sequence: | |
| CCGTTTTAAGCCAGCCCCGACACCCGCCAACACCCGCTGACGCGCCCTGACGGGCTTGTCTG | (Seq ID NO: 14) |
| CTCCCGGCATCCGCTTACAGACAAGCTGTGACCGTCTCCGGGAGCTGCATGTGTCAGAGGTT | |
| TTCACCGTCATCACCGAAACGCGCGAGACGAAAGGGCCTCGTGATACGCCTATTTTTATAGG | |
| TTAATGTCATGATAATAATGGTTTCTTAGACGTCAGGTGGCACTTTTCGGGGAAATGTGCGC | |
| GGAACCCCTATTTGTTTATTTTTCTAAATACATTCAAATATGTATCCGCTCATC | |

### EXAMPLES

The following examples illustrate the practice of the present invention but should not be construed as limiting its scope.

### Example 1

### Preparation of Oligos

The oligonucleotides listed in Table 1 below were prepared for use in the following examples.

TABLE 1

Oligonucleotides

SEQ ID NO: 15

gac tct tca agc gca aga gcg ccc aat acg ca

SEQ ID NO: 16

AAT ggC TAg CAA Agg AgA AgA AC

SEQ ID NO: 17

TAA ggT TAT TTg Tag AgC TCA TCC ATg C

SEQ ID NO: 18

ATT ggT AgC TgT TTC CTg TgT gAA ATT gT

SEQ ID NO: 19

TTA ggT TAA gCC AgC CCC gAC ACC CgC CA

SEQ ID NO: 20

ggC ATg gCT AgC AAA ggA gAA gAA C

SEQ ID NO: 21

Cgg TTA TTT gTA gAg CTC ATC CAT gCC A

SEQ ID NO: 22

gCC TTA gCT gTT TCC TgT gTg AAA TTg TT

SEQ ID NO: 23

CCg TTT TAA gCC AgC CCC gAC ACC CgC CA

SEQ ID NO: 24

AAA ATg gCT AgC AAA ggA gAA gAA C

SEQ ID NO: 25

TTA ggT TAT TTg Tag AgC TCA TCC ATg CC

SEQ ID NO: 26

ATT TTC gAg CTg TTT CCT gTg TgA AAT Tg

SEQ ID NO: 27

TAA CCT TAA gCC AgC CCC gAC ACC CgC CA

SEQ ID NO: 28

CCA Tgg CTA gCA AAg gAg AAg AAC

SEQ ID NO: 29

gCC TTA TTT gTA gAg CTC ATC CAT gCC AT

SEQ ID NO: 30

ggT TAg CTg TTT CCT gTg TgA AAT TgT T

SEQ ID NO: 31

ggC TTT TAA gCC AgC CCC gAC ACC CgC CA

### Example 1A

### Ligation of Two Linear DNAs (PCR Reaction Products) by Sticky RICE

A 225 bp fragment of pUC 19 was amplified by the PCR using oligos Seq ID NO: 15 and Seq ID NO: 18, in a 100 ul reaction containing 1×Taq DNA polymerase buffer with MgCl2 (Promega), 100 pmoles each oligo, 0.2 mM final concentration of each dNTP (dGTP, dCTP, dATP, dTTP), 50 pg pUC 19 (Invitrogen) and 5U Taq DNA polymerase (Promega catalog # Ml 661). The reaction was heated to 94° C. for 3 minutes then cycled for 30 times through the following three temperatures: 94° C., 15 sec; 50° C. 30 sec; 72° C. 20 sec. After the final cycle, the reaction was incubated at 72° C. for 5 minutes.

A 770 bp DNA fragment of the GFP gene was amplified from the plasmid p30BGFPc3 (Shivprasad S., Pogue, G P, Lewandowski D J, Hidalgo J, Donson J, Grill L K, Dawson W O. 1999. Heterologous sequences greatly affect foreign gene expression in tobacco mosaic virus-based vectors. Virology 255(2) 312-323.) by the PCR using oligos Seq ID NO: 16 and Seq ID NO: 17, in a 100 ul reaction containing 1×Taq DNA polymerase buffer with MgCl2 (Promega), 100 pmoles each oligo, 0.2 mM final concentration of each dNTP (dGTP, dCTP, dATP, dTTP), and 5U Taq DNA polymerase (Promega catalog #M1661). The reaction was heated to 94° C. for 3 minutes then cycled for 30 times through the following three temperatures: 94° C., 20 sec; 50° C. 30 sec; 72° C. 45 sec. After the final cycle, the reaction was incubated at 72° C. for 5 minutes.

Following PCR the PCR products were purified from unincorporated dNTPs, oligonucleotides, buffer, etc using the StrataPrep PCR purification kit (Stratagene) using the manufacturers instructions. The purified PCR products were eluted from StrataPrep columns with dH₂O.

Approximately 50 ng of the two purified PCR reaction products were combined in a 10 ul reaction containing: 1× New England Biolabs (NEB) ligase buffer, 1.25 U T4 DNA pol (Novagen, LIC qualified), and 0.2 mM (each) dGTP and dCTP. The reaction was incubated at 37° C. for 30 minutes. Following the 37° C. treatment 0.5 ul of a mixture of 0.2Units/ul Gibco T4 DNA ligase and 1 Unit/ul T4 polynucleotide kinase (Novagen) in 1×NEB ligase buffer was added, the reaction mixed by pipetting. The reaction was then allowed to proceed for approximately 1 hour at room temperature.

The T4 DNA polymerase and dNTP treatment generated 5′ overhangs on the two PCR products as described in the table below:

!225 bp PCR Product? 770 bp PCR Product

| | | |
|---|---|---|
| PCR primers | 308-351 | 349-350 |
| used | | |
| ds PCR prod | 5′gac tct--------cc aat | 5′aat ggc-----------acc tta |
| | ctg aga--------gg tta | tta ccg-----------tgg aat |
| Post T4 pol treat | 5′gac tct----------cc | 5′aat ggc-----------acc |
| with dGTP/dCTP | ctg aga----------gg tta | ccg-----------tgg aat |

The 5′ overhangs of the two polynucleotides in bold are complementary to each other.

After incubating at room temperature, the reaction was analyzed by electrophoresis through a 2% Agarose gel in 1×TAE buffer, stained with EtBr and photographed under UV light.

### Example 1B:

Example 1B was performed using the substantially the same procedures of Example 1A with the following exceptions: The 225 bp product from pUC 19 was generated with oligos Seq ID NO: 15 and Seq ID NO: 22. The 770 bp product (GFP gene) was amplified with PCR primers Seq ID NO: 20 and Seq ID NO: 21. And the T4 DNA polymerase treatment contained 0.2 mM (each) dATP and dTTP, in place of dGTP and dCTP.

The T4 DNA polymerase and dNTP treatment generated 5′ overhangs on the two PCR products as described in the table below:

!225 bp PCR Product? 770 bp PCR Product

| | | |
|---|---|---|
| PCR primers | 308-355 | 353-354 |
| used | | |
| ds PCR prod | 5′gac tct---------taa ggc | 5′ggc atg---------taa ccg |
| | ctg aga---------att ccg | ccg tac---------att ggc |
| Post T4 pol treat | 5′gac tct----------taa | 5′ggc atg---------taa |
| with dATP/dTTP | tg aga----------att ccg | tac--------att ggc |

The 5′ overhangs of the two polynucleotides in bold are complementary to each other.

After incubating at room temperature, the reaction was analyzed by electrophoresis through a 2% Agarose gel in 1×TAE buffer, stained with EtBr and photographed under UV light.

### Example 1C

Example 1C was performed using substantially the same procedures of Example 1A with the following exceptions: The 225 bp product from pUC 19 was generated with oligos Seq ID NO: 15 and Seq ID NO: 26. The 770 bp product (GFP gene) was amplified with PCR primers Seq ID NO: 24 and Seq ID NO: 25

The T4 DNA polymerase and dNTP treatment generated 5′ overhangs on the two PCR products as described in the table below:

| | |
|---|---|
| 225 bp PCR Product | 770 bp PCR Product |

| | | |
|---|---|---|
| PCR primers | used 308-359 | 357-358 |
| Ds PCR prod | 5′gac tct----------gaa aat | 5′aaa atg g--------acc taa |
| | ctg aga----------ctt tta | ttt tac c--------tgg att |
| Post T4 pol treat | 5′gac tct----------g | 5′aaa atg g-------acc |
| with dGTP/dCTP | ctg aga----------ctt tta | c c-------tgg att |

The 5′ overhangs in bold will be complementary to each other.

After incubating at room temperature, the reaction was analyzed by electrophoresis through a 2% Agarose gel in 1×TAE buffer, stained with EtBr and photographed under UV light.

### Example 1D

Example 1D was performed using substantially the same procedures of Example 1B with the following exceptions: The 225 bp product from pUC 19 was generated with oligos Seq ID NO: 15 and Seq ID NO: 30. The 770 bp product (GFP gene) was amplified with PCR primers Seq ID NO: 28 and Seq ID NO: 29.

The T4 DNA polymerase and dNTP treatment generated 5′ overhangs on the two PCR products as described in the table below:

| | |
|---|---|
| 225 bp PCR Product | 770 bp PCR Product |

| | | |
|---|---|---|
| PCR primers used | 308/363 | 361-362 |
| ds PCR prod | 5′gac tct----------aa cc | 5′cc atg---------taa ggc |
| | ctg aga----------tt gg | gg tac---------att ccg |
| Post T4 pol treat | 5′gac tct----------aa | 5′cc atg---------taa |
| with dATP/dTTP | tg aga----------tt gg | tac---------att ccg |

The 5′ overhangs of the two polynucleotides in bold are complementary to each other.

After incubating at room temperature, the reaction was analyzed by electrophoresis through a 2% Agarose gel in 1×TAE buffer, stained with EtBr and photographed under UV light.

Results of Examples 1A-1D:

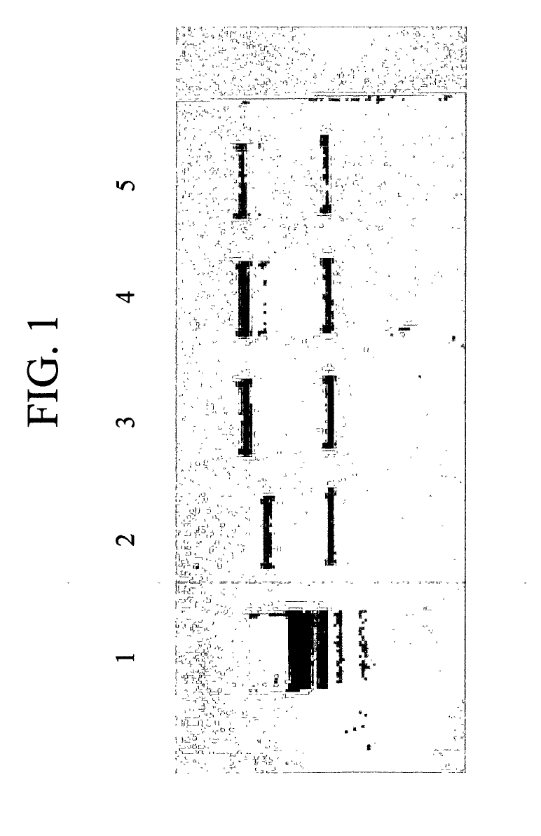

In each of the examples 1A-1D (depicted in FIG. 1), the desired result would be for the ligation of the 225 and 770 bp products to generate a DNA fragment of about 1000 bp in size. In FIG. 1, Lane 1=Promega 100 bp DNA molecular weight ladder; lane 2=Ex 1A reaction, lane 3=Ex 1B reaction; lane 4=Ex 1C reaction; lane 5=Ex 1D reaction.

FIG. 1 is a photograph of a gel that shows the relative efficiency of ligation under these conditions and is as follows: B=C>D>>A. The relative ligation efficiency in this experiment paralleled the calculated melting temperature (Tm) for the predicted sticky ends, using the formula Tm=2(A/T)+4 (G/C), where Tm is in degrees C. Using this formula the predicted Tms for the various sticky ends are: Ex 1B (12° C.), Ex 1C (10° C.), Ex 1D (8° C.), Ex 1A (6° C.).

If desired the ligation reaction step of the sticky RICE reaction can be performed at 16° C. and/or for extended periods of time to increase the final yield of ligated product.

### Example 2

### Construction of a Recombinant Plasmid Using Sticky RICE

Vector Preparation.

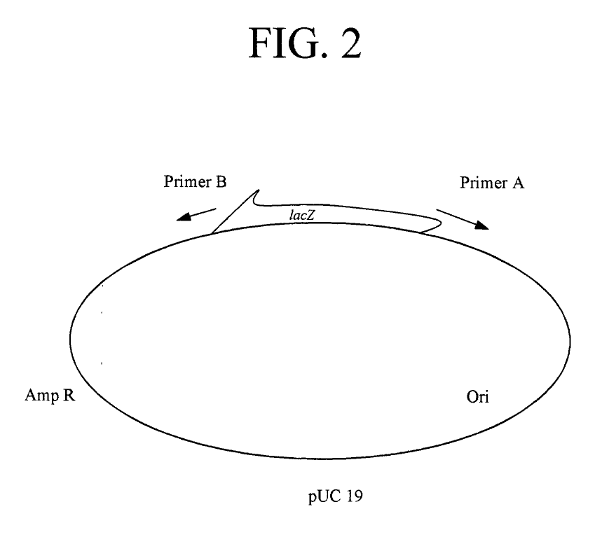

Several modifications of pUC 19 were generated using the polymerase chain reaction. Primer pairs were designed so that an “inverse PCR” reaction of the pUC 19 DNA template would generate a ds DNA of about 2.6 kb in size. The 5′ end of one primer of each pair will sit just upstream of the ATG start codon of the lac alpha peptide ORF, with its 3′ end proximal to the Lac promoter. The 5′ end of the other primer of each pair will sit just downstream of the stop codon of the lac alpha peptide with the 3′ end proximal to the promoter for the ampicillin resistance gene. FIG. 2 shows the plasmid pUC 19 and the relative locations and directions of the primers used for amplification of pUC 19 by PCR for experiments 2A-2D. Primers are represented by the solid arrows, 5′ (blunt end of arrow) to 3′ (point of arrow). Primers (Seq ID NOS:) 18, 22, 26 and 30 hybridize to pUC 19 at the location identified by primer B in FIG. 2. Primers (Seq ID NOS:) 19, 23, 27 and 31 hybridize to pUC 19 at the location identified by primer A in FIG. 2.

Specifically, FIG. 2 shows the plasmid pUC 19 and the relative locations and directions of the primers used for amplification of pUC 19 by PCR for experiments 2A-2D. Primers are represented by the solid arrows, 5′ prime (blunt end of arrow) to 3 prime (point of arrow). Primers (Seq ID NOS:) 18, 22, 26 and 30 hybridize to pUC 19 at the location identified by primer B in figure above. Primers (Seq ID NOS:) 19, 23, 27 and 31 hybridize to pUC 19 at the location identified by primer A in FIG. 2.

The pUC 19 plasmid was amplified by the PCR using oligos Seq ID NO: 18 and Seq ID NO: 19, in a 100 ul reaction using the 1×buffer described by Barnes (Barnes WM. 1994. PCR amplification of up to 35 kB DNA with high fidelity and high yield from lambda bacteriophage templates. PNAS 91(6) 2216-20). 100 pmoles each oligo, 0.2 mM final concentration of each dNTP (dGTP, dCTP, dATP, dTTP), 100 pg pUC 19 (Invitrogen) and 3.5U Taq DNA polymerase (Promega catalog # M1661) and 0.035 Units Pfu Turbo DNA polymerase (Stratagene) were combined to form a reaction mixture. The reaction was heated to 94° C. for 3 minutes then cycled for 30 times through the following three temperatures: 94° C., 30 sec; 50° C. imin; 72° C. 3 min. After the final cycle, the reaction was incubated at 72° C. for 5 minutes.

Separate PCR reactions of pUC 19 DNA template were set up as above, using the following primer pairs: SEQ ID NO: 22 and SEQ ID NO: 23; SEQ ID NO: 26 and SEQ ID NO: 27; SEQ ID NO: 30 and SEQ ID NO: 31.

The PCR reactions were then cooled to 37° C. and 20 units of the restriction endonuclease DpnI (New England Biolabs) was added to digest the dam methylated pUC 19 template DNA and the reaction incubated at 37° C. for 90 minutes.

Following the DpnI treatment, the PCR products were purified from unincorporated dNTPs, oligonucleotides, buffer, etc., using the StrataPrep PCR purification kit (Stratagene) using the manufacturers instructions. Each purified PCR product was eluted from StrataPrep columns with dH₂O.

The various 2.6 kb pUC 19 PCR products were used in sticky RICE reactions with GFP PCR products from experiments 1A-1D (Examples 1A, 1B, 1C and 1D) as described below:

### Example 2A

Approximately 20 ng purified pUC 19 PCR product and 12 ng of a 770 bp GFP PCR product were combined in a 10 ul reaction containing IX NEB ligase buffer, 0.2 mM (each) dGTP and dCTP, 0.8 Units T4 DNA pol (Novagen LIC qualified), 0.06 Units Gibco T4 DNA ligase and 0.6 Units T4 polynucleotide kinase (Novagen). The reaction was incubated at room temperature for approximately 2.5 hours and then 1 ul was transformed into DH5a chemically competent E. coli cells (Gibco). Approximately ⅕th of the transformed cells were then plated onto individual LB Agar plates containing 100 ug/ml of Ampicillin and plates were incubated overnight at 37° C.

The following day, individual colonies were picked into 2 ml samples of LB broth containing 100 ug/ml Ampicillin, and grown overnight in an incubator-shaker at 37° C. at approx 250-300 rpm. Plasmid DNA was prepared from these overnight liquid cultures using the StrataPrep Plasmid Miniprep kit (Stratagene) according to the manufacturer's instructions.

To screen for the presence of a GFP sized DNA insert in plasmids, individual DNA samples of 2 ul were digested with the restriction endonuclease AflIII (NEB) according to manufacturers suggestions. AflIII will cleave once in the pUC 19 backbone, and once in the GFP insert, to release a DNA fragment of approximately 700 bp. AflIII digested DNA samples were analyzed by electrophoresis through a 1% agarose gel in 1×TAE buffer. The gel was then stained with Ethidium bromide staining and photographed under UV light. The presence of a insert band of about 700 bp indicated the presence of an insert in a plasmid.

The T4 DNA polymerase and dNTP treatment generated 5′ overhangs on the two PCR products as described in the table below:

| | |
|---|---|
| pUC 19 PCR Product | 770 bp PCR Product |

| | | |
|---|---|---|
| PCR primers | 351-352 | 349-350 |
| used | | |
| ds PCR prod | 5′tta ggt--------cc aat | 5′aat ggc-----------acc tta |
| | aat cca-------gg tta | tta ccg-----------tgg aat |
| Post T4 pol treat | 5′tta ggt--------cc | 5′aat ggc-----------acc |
| with dGTP/dCTP | cca--------gg tta | ccg-----------tgg aat |

### Example 2B

The sticky RICE reaction of Ex 2B was set up using substantially the same procedures of 2A except that 0.2 mM (each) dATP and dTTP were used in place of 0.2 mM dGTP and dCTP, and PCR products of oligos (SEQ ID NO: 22)/(SEQ ID NO: 23) and (SEQ ID NO: 20)/(SEQ ID NO: 21) were used.

The T4 DNA polymerase and dNTP treatment generated 5′ overhangs on the two PCR products as described in the table below:

| | |
|---|---|
| pUC 19 PCR product | 770 bp PCR Product |

| | | |
|---|---|---|
| PCR primers | 355-356 | 353-354 |
| used | | |
| ds PCR prod | 5′ccg ttt---------taa ggc | 5′ggc atg---------taa ccg |
| | ggc aaa--------att ccg | ccg tac---------att ggc |
| Post T4 pol treat | 5′ccg ttt---------taa | 5′ggc atg---------taa |
| with dATP/dTTP | ∩aaa--------att ccg | tac--------att gg |

The 5′ overhangs in bold are complementary to each other. The 5′ overhangs underlined are complementary to each other.

### Example 2C

The sticky RICE reaction of Ex 2C was set up using substantially the same procedures of 2A except that the PCR products of (SEQ ID NO: 26)/(SEQ ID NO: 27) and (SEQ ID NO: 24)/(SEQ ID NO: 25) were used.

The T4 DNA polymerase and dNTP treatment generated 5′ overhangs on the two PCR products as described in the table below:

!pUC 19 PCR Product? 770 bp PCR Product

| | | |
|---|---|---|
| PCR primers | 359-360 | 357-358 |
| used | | |
| ds PCR prod | 5′taa cct----------gaa aat | 5′aaa atg g--------acc taa |
| | att gga----------ctt tta | ttt tac c-------tgg att |
| Post T4 pol treat | 5′taa cct----------g | 5′aaa atg g-------acc |
| with dGTP/dCTP | gga----------ctt tta | c c-------tgg att |

The 5′ overhangs in bold will be complementary to each other. The 5′ overhangs underlined will be complementary to each other.

### Example 2D

The sticky RICE reaction of Ex 2D was set up using substantially the same procedures of 2B except that PCR products of oligos (SEQ ID NO: 30)/(SEQ ID NO: 31) and (SEQ ID NO: 28)/(SEQ ID NO: 29) were used.

The T4 DNA polymerase and dNTP treatment generated 5′ overhangs on the two PCR products as described in the table below:

!pUC 19 PCR product? 770 bp PCR Product

| | | |
|---|---|---|
| PCR primers used | 363-364 | 361-362 |
| ds PCR prod | 5′ggc ttt----------aa cc | 5′cc atg---------taa ggc |
| | ccg aaa--------tt gg | gg tac---------att ccg |
| Post T4 pol treat | 5′ggc ttt----------aa | 5′cc atg---------taa |
| with dATP/dTTP | aaa---------tt gg | tac---------att ccg |

The 5′ overhangs in bold are complementary to each other. The 5′ overhangs underlined are complementary to each other.

Results of Examples 2A-2D:

| Rxn | # colonies/plate | % colonies with inserts |
|---|---|---|
| 2A | 6 | 2/8 (25%) |
| 2B | ca. 250 | 23/24 (96%) |
| 2C | >700 | 23/32 (72%) |
| 2D | >700 | 21/32 (66%) |

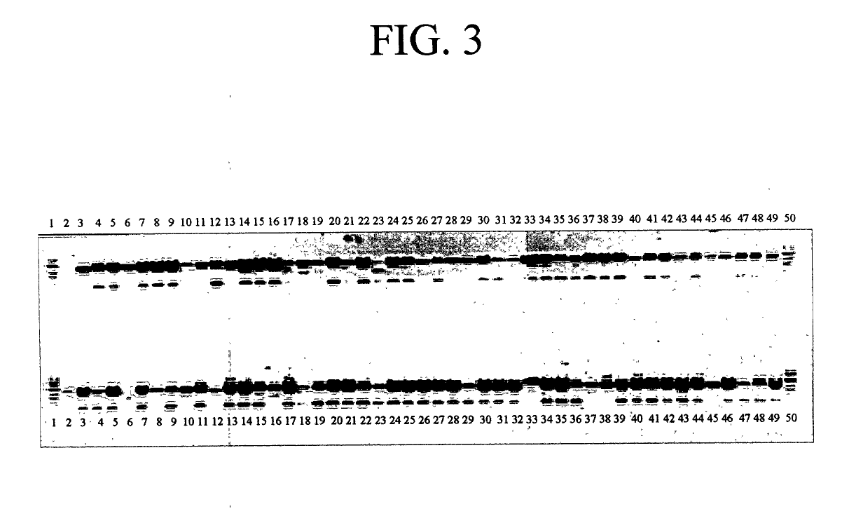

FIG. 3 is a photograph of a gel of the experimental results of Example 2 (Sticky RICE cloning of inserts into pUC using T4 DNA ligase). Plasmids from the cloning reactions described in examples 2A-2D were digested with AflIII to screen for the presence of a GFP insert. Plasmids with the GFP PCR product insert are expected to release a DNA fragment of approx. 700 bp after digestion with AflIII. Lanes of gel are numbered from left to right. The far right and left lanes of both top and bottom half of gel contain 1 Kb DNA ladders from New England Biolabs. The bottom half of the gel contains 8 isolates of clones from experiment 2A (lanes 2, 4, 6, 8, 10, 12, 14 and 16). Isolates of experiment 2B cloning are in the bottom of the gel, odd numbered lanes 3-17, and lanes 18-33 (24 isolates total). Isolates of experiment 2C are in lanes 34-49 in the bottom of the gel, and lanes 2-17 in the top of the gel (32 isolates total). Isolates of experiment 2D are in lanes 18-49 in the top of the gel (32 isolates total). The approx 700 bp band is the lower of the two bands in lane 3 in the bottom of gel.

Experiment 3

Sticky RICE with E. coli DNA Ligase

Rxns 3A-3D were performed as described for 2A-2D, respectively, with the following modifications:

1. The reactions were performed in IX E. coli DNA ligase buffer (New England Biolabs) supplemented with rATP to a final concentration of 1 mM.

2. Each 10 ul sticky RICE reaction contained 0.7 Units T4 DNA pol (Novagen LIC qualified), 6.6 Units E. coli DNA ligase (NEB) and 1.1 Units T4 polynucleotide kinase (NEB).

3. Reactions proceeded at room temperature overnight. One microliter of each sticky RICE reaction (3A-3D) was transformed in to DH5a chemically competent E. coli cells (Gibco), according to manufacturers instructions.

Approximately ½ of the entire sample of transformed cells were plated on LB Agar plates containing 100 ug/ml Ampicillin and plates were incubated overnight at 37° C.

The following day, individual colonies were picked into 2 ml samples of LB broth containing 100 ug/ml Ampicillin, and grown overnight in an incubator-shaker at 37° C. at approx 250-300 rpm. Plasmid DNA was prepared from these overnight liquid cultures using the StrataPrep Plasmid Miniprep kit (Stratagene) according to the manufacturer's instructions.

To screen for the presence of a GFP sized DNA insert in plasmids, individual DNA samples of 2 ul were digested with the restriction endonuclease AflIII (NEB) according to manufacturers suggestions. AflIII will cleave once in the pUC 19 backbone, and once in the GFP insert, to release a DNA fragment of approximately 700 bp. AflIII digested DNA samples were analyzed by electrophoresis through a 1% agarose gel in 1×TAE buffer. The gel was then stained with Ethidium bromide staining and photographed under UV light. The presence of a insert band of about 700 bp indicated the presence of an insert in a plasmid.

Results of Transformation Reaction.

| Reaction | # colonies | % clones with insert |
|---|---|---|
| 3A | 0 | NA |
| 3B | >500 | 8/8 (100%) |
| 3C | 0 | NA |
| 3D | >500 | 7/8 (88%) |

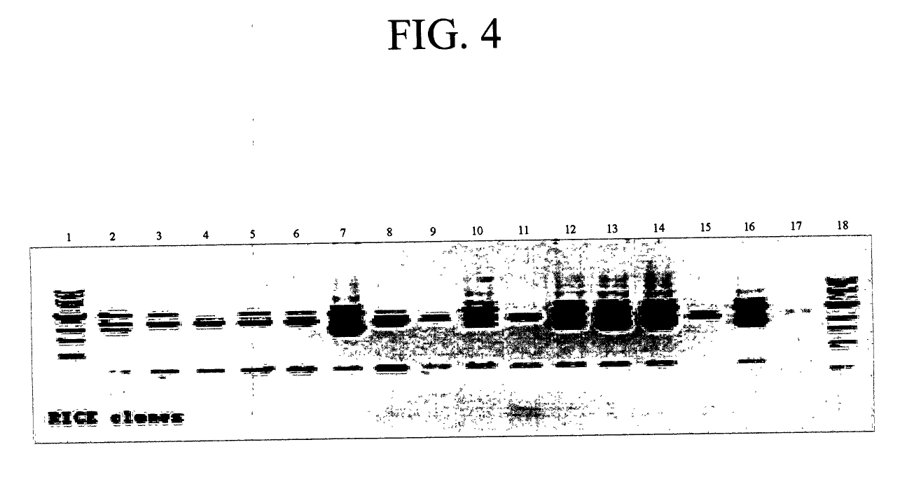

FIG. 4 is a photograph of a gel of the experimental results of Example 3, (Sticky RICE cloning of inserts into pUC using E. coli ligase). Lanes of gel are numbered left to right. Lanes 1 and 18 contain a 1 Kb DNA ladder from New England Biolabs. Lanes 2-9 contain 8 isolates of Exp 3B. Lanes 10-17 contain 8 isolates of Exp 3D. The approximately 700 bp band released upon digestion with AflIII is the lower of the two bands in lane 4, for example. The AflIII restriction digest did not go to completion in all samples due to the large amount of DNA contained in some samples. This resulted in a partial DNA digest for many samples, though the unique 700 bp band can still be detected.

### Example 4

### Sticky RICE Cloning of PCR Fragments into a SapI Cut Vector

A tobacco mosaic virus based plant expression vector (Shivrasad et al 1999) was modified to contain 2 unique SapI restriction sites.

The typeII S restriction enzyme SapI, cleaves ds DNA to leave 3 nt 5′ overhangs. A TMV based cDNA clone in a plasmid was generated with two SapI sites, such that SapI digested plasmid would be compatible with a sticky RICE cloning strategy of PCR products. This was accomplished as follows:

Mutation of the SapI Site in the pUC DNA Backbone of a p30BGFPc3 Vector (pCLONE5).

Since the desired plasmid will only have SapI sites in the TMV cDNA insert, the SapI site, which exists in the pUC plasmid backbone, must first be mutated. This was accomplished by using the PCR to amplify a portion of pCLONE5 DNA with oligos SEQ ID NO: 15 and 30B7093F, spanning from near the 3′ end of the virus cDNA insert, to just downstream of the SapI site in the pUC backbone. The resulting PCR product was digested with the restriction enzymes KpnI and EarI, electrophoresed thru an agarose gel, and a ds DNA fragment of approximately 300 bp was isolated from the gel. This DNA fragment was then ligated to the approximately 10 Kb DNA fragment of a pCLONE5 digested with SapI and KpnI. The resulting plasmid was named pC5DSapI.

pC5DSapI was digested with NcoI and PmlI (which cleave at nts 5459 and 6536, respectively, in pC5DSapI). The resulting 9.3 kb fragment was gel isolated.

Using pCLONE5 template DNA, a DNA fragment of approximately 700 bp was amplified using oligos SEQ ID NO: 34 and 30B5123F. Oligo SEQ ID NO: 34 will generate a SapI site fused to TMV cDNA vector sequences.

A cDNA insert of human thymosin cDNA was amplified and cloned into pcDNA3.l V5 His topo cloning vector (Invitrogen). The thymosin insert and V5 His6 sequence was PCR amplified from this plasmid with oligos SEQ ID NO: 32 and a thymosin specific (forward) primer containing a PacI site near its 5′ end. This PCR product was digested with PacI and SalI and ligated into PacI-XhoI digested pCLONE5, in order to insert the V5 epitope and His6 coding sequence into the TMV cDNA. From this plasmid, the V5 His6 sequence and downstream TMV vector sequence was amplified by PCR with oligos SEQ ID NO: 35 and 30B6543R (a fragment of about 200 bp). Oligo SEQ ID NO: 35 will generate a SapI site just upstream of the V5 epitope coding sequence.

These two PCR fragments were joined together in a sticky RICE reaction by combining approximately equamolar amounts of PCR products with T4 DNA polymerase and dGTP/dCTP blocking nucleotides, T4 polynucleotide kinase, and T4 ligase in 1×NEB ligase buffer, under conditions similar to those described in Example 1.

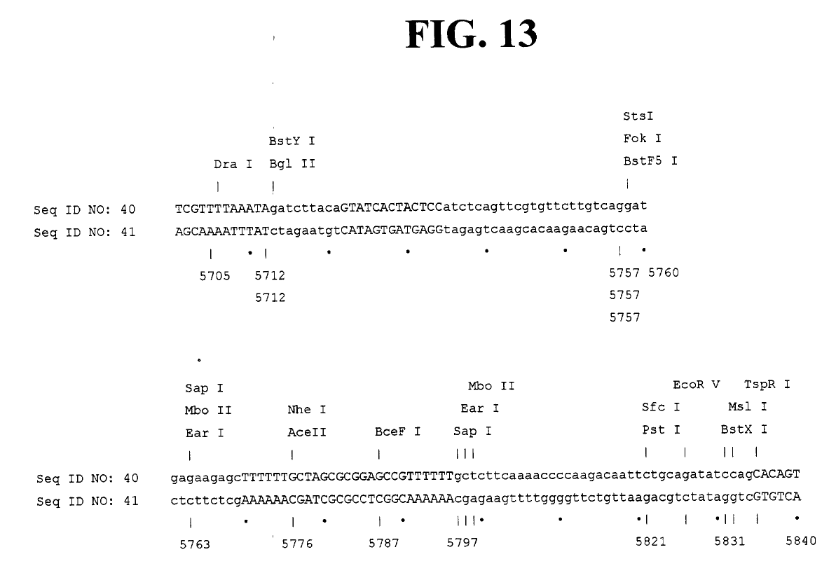

After the Sticky RICE reaction, the reaction was extracted with phenol and chloroform and the DNA precipitated with ammonium acetate and ethanol using standard conditions. The precipitated DNA was washed with 70% EtOH, briefly dried and ultimately resuspended in dH₂O. This DNA was then digested with the restriction endonucleases NcoI and PmlI. The digested DNA was electrophoresed through an agarose gel and the approx 570 bp fragment gel isolated. This approx. 570 bp fragment was then ligated into the approximately 10 Kb fragment of NcoI-PmlI digested pC5DSapI. This generated the plasmid pLSB 1176 (see FIG. 13), which has the following (relevant) polylinker sequence:

```
                                                                                                                StsI
                      BstY I Fok I
        Dra I Bgl II BstF5 I
        | | |
TCGTTTTAAATAgatcttacaGTATCACTACTCCatctcagttcgtgttcttgtcaggat
AGCAAAATTTATctagaatgtCATAGTGATGAGGtagagtcaagcacaagaacagtccta
        | • | • • • • | •
        5705 5712 5757 5760
                      5712 5757
                                                                                                            5757
    Sap I Mbo II EcoR V TsPR I
    Mbo II Nhe I Ear I Sfc I Msl I
    Ear I AceII BceF I Sap I Pst I BstX I
    | | | ||| | | || |
gagaagagcTTTTTTGCTAGCGCGGAGCCGTTTTTTgctcttcaaaaccccaagacaattctgcagatatccagCACAGT
ctcttctcgAAAAAACGATCGCGCCTCGGCAAAAAAcgagaagttttggggttctgttaagacgtctataggtcGTGTCA
    | • | • | • |||• • •| | •|| | •
5763 5776 5787 5797 5821 5831 5840
```

Note the position of the 2 SapI sites (at approx nt 5763 and 5797). The SapI site at 5763 cuts to its left, leaving an TAC5′ overhang on the bottom strand and the SapI site at 5797 cuts to its right, leaving a 5′ AAA overhang on the top strand.

For pLSB 1176, the following Oligos were used:

SEQ ID NO: 15

gactcttcaa gcgcaagagc gcccaatacg ca

SEQ ID NO: 32

CggCTggTCg ACgCGggTTT A

SEQ ID NO: 33

AACTCAATgg TgATg

SEQ ID NO: 34

AAAAAAgCTC TTCTCATCCT gACAAgAACA CgAACTgAgA T

SEQ ID NO: 35

TTTTTTgCTC TTCAAAACCC CAAgACAATT CTgCAgATAT CCAg

SEQ ID NO: 36

TTTTTTgCTA gCgCggAgCC gTTTTTTgCT CTTCAAAACC
CCAAgACA

SEQ ID NO: 37

(30B 5123F) TgTTTAgCCg gTTTggTCgT

SEQ ID NO: 38

(30B 7093F) TTgATCCgTT gATCACggCg T

SEQ ID NO: 39

(30B 6543R) CACTATgCgT TATCgTACgC A

After digestion with SapI, the vector pLSB 1176 was electrophoresed thru a 1% agarose in 1×TAE gel and isolated from the gel.

An insert was prepared for this vector by PCR of the GFPc3 gene from pCLONE5 with the oligos:

SEQ ID NO: 46

ATg CCC gCT AgC AAA ggA gAA gAA CTT TTC
and

SEQ ID NO: 47

TTT CCC ITT gTA gAg CTC ATC CAT gCC ATg

(SEQ ID NO: 46 is the Forward direction oligo for GFP and SEQ ID NO: 47 is the Reverse direction oligo for GFP) which would generate a PCR product of about 770 bp which, when treated with dGTP would give sticky ends compatible with the approximately 10 kb SapI digested pLSB 1176 vector fragment (see table below).

| | Insert DNA ends (SEQ ID NO: 40) | SapI cut Vector DNA ends+HZ,1 45 |
|---|---|---|
| Treat with T4 | atg ccc GCT AGC--- tac aaa ggg aaa | --cagg aaa ccc--- |
| DNA pol and | tac ggg cga tcg-- atg ttt ccc ttt | --gtcc tac ggg--- |
| dGTP | | |
| post T4+dGTP | atg ccc GCT AGC--- tac aaa ggg | --cagg aaa ccc--- |
| treat | ggg cga tcg-- atg ttt ccc ttt | --gtcc tac ggg--+TZ,1 45 |

Note that in FIG. 13 the sticky ends of the SapI cut vector are unchanged by the T4+dGTP treatement. 24 ng of SapI cut pLSB 1176 vector and 4 ng of PCR product were combined in a 10 ul rxn containing 0.2 mM dGTP in 1×NEB ligase buffer and 1.25 Units Novagen LIC qualified T4 DNA pol. Reaction was incubated at 37° C. for about 30-40 min, and 0.5 Units of Gibco DNA ligase added to the reaction. The reaction was then mixed by pipetting and incubated at 16° C. overnight. One microliter of the overnight ligation mix was transformed into DH5a chemically competent E. coli cells (Gibco). After transformation the cells were plated on LB agar plates containing 100 ug/ml ampicillin. Plates were incubated overnight at 37° C. The next day 12 colonies were picked from the plates and used to inoculate 2 ml samples of LB broth with 100 ug/ml ampicillin. DNA was prepared from liquid cultures using the strataprep plasmid DNA purification system (Stratagene). DNA samples were screened for the presence of the GFP based PCR insert by digestion with the restriction endonuclease HindIII followed by agarose gel electrophoresis of the digested DNAs. Of 12 plasmids screened in this manner, all 12 had inserts.

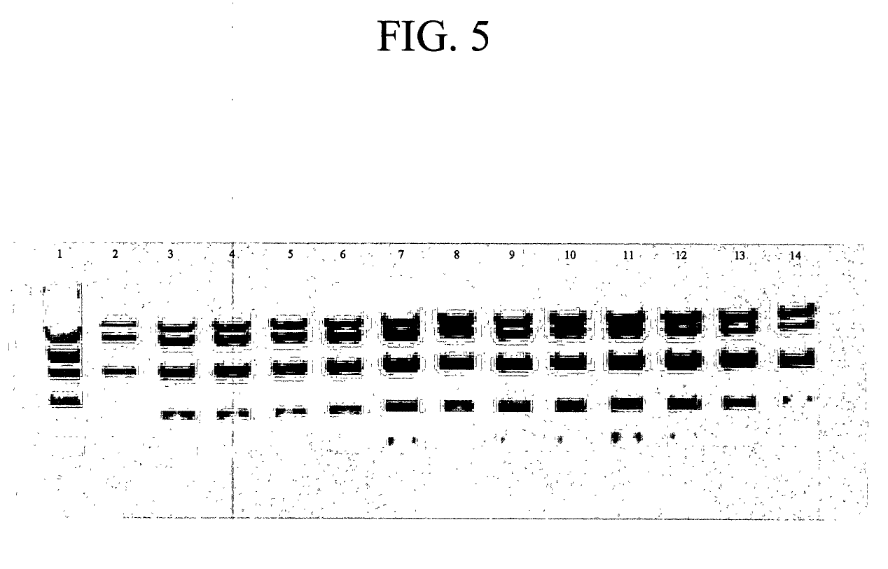

FIG. 5 is a photograph of a gel of the experimental results for Example 4 (Sticky RICE cloning of GFP PCR product into SapI digested vector DNA—pLSB 1176). The lanes of gel are numbered from left to right. Lane 1 contains a 1 Kb DNA ladder from New England Biolabs. Lane 2 contains HindIII digested pCLONE 5 control DNA (which already contains the GFP insert). Clones of interest from the experiment will generate the same HindIII restriction enzyme digestion pattern as the pCLONE 5 control DNA. Lanes 3-14 contain isolates 1-12 of Experiment 4. Note all show HindIII restriction enzyme digestion patterns equivalent to the control.

### Example 5

### The effect of Relative Polymerase, Kinase and Ligase Concentrations on Sticky RICE Reactions

Experiments were conducted to examine the effect of relative polymerase and kinase and ligase concentrations on the efficiency of the Sticky RICE reaction. Various concentrations of kinase and polymerase were assembled, keeping the T4 DNA ligase (Gibco/BRL) concentration constant at 0.25 units per reaction. Concentrations of T4 DNA polymerase (Novagen LIC qualified) from 2.5 U to 0 U were tested. T4 DNA kinase (New England Biolabs) concentrations between 2.5 and 0 U were also tested. Each reaction consisted of 7.8 ng of a 225 bp PCR product, 25 ng of a 725 bp PCR product, 1 ul 10×ligase buffer (New England Biolabs), 1 ul 2 mM dATP/dTTP stock solution in a final volume of 12.4 ul. Sources of enzymes were as follows: T4 DNA polymerase (Novagen, LIC qualified), T4 polynucleotide kinase (New England Biolabs), T4 DNA ligase (Gibco/BRL).

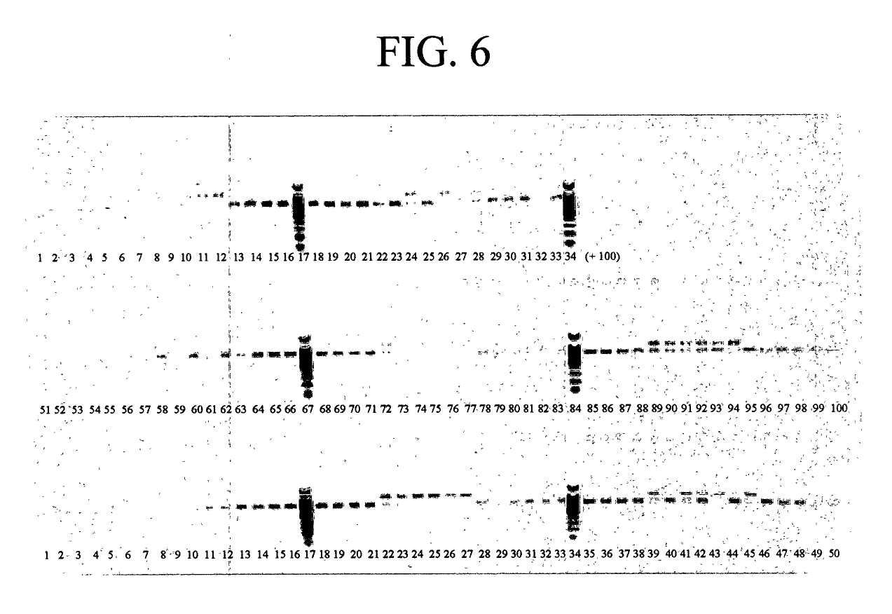

Two different temperatures were tested in this Sticky RICE experiment. Reactions were either incubated at 37° C. for 10 minutes followed by 2 hours at room temp, or were treated only at room temperature for 2 hours. The Sticky RICE reactions were terminated by incubation at 75° C. for 10 minutes. Reactions were then electrophoresed on a 1.5% agarose gel (in 1×TAE buffer, Sambrook et al Molecular cloning manual). After electrophoresis DNA was visualized by staining the gel with ethidium bromide (EtBr) and photographed under long wave UV light, as per standard conditions (Sambrook et al.). To estimate the efficiency of the Sticky RICE reaction the staining intensity of the approx 950 bp band (formed by the joining of the 225 and 725 bp DNA fragments, for example the “top” band in Lane 10 of FIG. 6) relative to the staining intensity of the 725 pb product was compared.

The 225 and 725 bp DNA fragments used in this Sticky RICE experiment were obtained by amplifying portions of the plasmid pUC 19 (using Pfu DNA polymerase from Stratagene Corp. and standard PCR reaction conditions, as suggested by the manufacturer) with the following primers:

PCR Primers Used:

SEQ ID NO: 15

5′ gactcttcaa gcgcaagagc gcccaatacg ca 3′

SEQ ID NO: 42

5′ ggcttagctg tttcctgtgt gaaattgtt 3′

SEQ ID NO: 43

5′ gccatggcta gcaaaggaga agaa c 3′

SEQ ID NO: 21

5′ cggttatttg tagagctcat ccatgcca 3′

The sequences of the 225 and 725 bp DNAs is presented below (225 Seq and 775 Seq)

225 Seq:
SEQ ID NO: 44
GACTCTTCA AGCGCAAGAGCG CCCAATACGCAA ACCGCCTCTCCC CGCGCGTTCGCC GATTCATTAATG CAGCTG
GCACGACAG GTTTCCCGACTG GAAAGCGGGCAG TGAGCGCAACGC AATTAATGTGAG TTAGCTCACTCA TTAGGC
ACCCCAGGC TTTACACTTTAT GCTTCCGGCTCG TATGTTGTGTGG AATTGTGAGCGG ATAACAATTTCA CACAGG
AAACAGCTA AGCC
725 Seq:
SEQ ID NO: 45
GCCATGAGT AAAGGAGAAGAA CTTTTCACTGGA GTTGTCCCAATT CTTGTTGAATTA GATGGTGATGTT AATGGG
CACAAATTT TCTGTCAGTGGA GAGGGTGAAGGT GATGCAACATAC GGAAAACTTACC CTTAAATTTATT TGCACT
ACTGGAAAA CTACCTGTTCCA TGGCCAACACTT GTCACTACTTTC TCTTATGGTGTT CAATGCTTTTCA AGATAC
CCAGATCAT ATGAAACGGCAT GACTTTTTCAAG AGTGCCATGCCC GAAGGTTATGTA CAGGAAAGAACT ATATTT
TTCAAAGAT GACGGGAACTAC AAGACACGTGCT GAAGTCAAGTTT GAAGGTGATACC CTTGTTAATAGA ATCGAG
TTAAAAGGT ATTGATTTTAAA GAAGATGGAAAC ATTCTTGGACAC AAATTGGAATAC AACTATAACTCA CACAAT
GTATACATC ATGGCAGACAAA CAAAAGAATGGA ATCAAAGTTAAC TTCAAAATTAGA CACAACATTGAA GATGGA
AGCGTTCAA CTAGCAGACCAT TATCAACAAAAT ACTCCAATTGGC GATGGCCCTGTC CTTTTACCAGAC AACCAT
TACCTGTCC ACACAATCTGCC CTTTCGAAAGAT CCCAACGAAAAG AGAGACCACATG GTCCTTCTTGAG TTTGTA
ACAGCTGCT GGGATTACACAT GGCATGGATGAA CTATACAAATGA CCG

During the Sticky RICE reaction the 3′ ends of the PCR products will be removed by the 3′ to 5′ exonuclease activity of the DNA polymerase, until the appropriate template nucleotide is reached. In this example the DNA is treated with T4 DNA polymerase in the presence of dATP and dTTP so that 5′ overhangs consist of only of G and C residues as described below:

| | 225 bp PCR product | 726 bp PCR product |
|---|---|---|
| | from pUC19 | from p30BGFPc3 |
| PCR primers used: | 308-397 | 398-354 |
| ds PCR product: | 5′gac tct ----- taa gcc | 5′gcc atg ----- taa ccg |
| | ctg aga ----- att cgg | cgg tac ----- att ggc |
| Post T4 pol treat | 5′gac tct ----- taa | 5′gcc atg ----- taa |
| with dATP/dTTP | tg aga -----att cgg | tac ----- att ggc |

*The 5′ overhangs in bald will be complementary to each other.

The amounts of polymerase, kinase and ligase in each reaction, and the gel loading is presented in Table 1. The gel image is shown in FIG. 6, below.

Note that not all combinations of polymerase, kinase and ligase resulted detectable amounts of recombinant DNA joining in this experiment. The experimental results demonstrate that “too much” or “too little” kinase or polymerase can result in inefficient DNA joining reactions. For example in Lane 1 a reaction with 2.5 U Pol, 2.5 U kinase and 0.25 U ligase did not result in any detecable DNA joining, but the reaction in Lane 5 which had 1.5 U Pol, 2.5 Kinase and 0.25 U ligase did result in detectable levels of DNA joining. Similarly the reaction in Lane 13 (0.1 U Pol, 2.5 U kinase and 0.25 U ligase) did not produce any detectable DNA joining, but the reaction in lane 63 (0.1 U Pol, 0.1 U kinase and 0.25 U ligase) did generate detectable levels of DNA joining. These results demonstrate that the relative levels of these three enzymatic activities dramatically effects the Sticky RICE reaction and that not all enzyme ratios lead to efficient joining of DNAs in vitro.

TABLE 1
Description of loading of gel shown in FIG. 6:

| Bottom Row, Left to Right. | | | | Middle Row, Left to Right | | | | Top Row, Left to Right | | | |
|---|---|---|---|---|---|---|---|---|---|---|---|
| Lane | Pol (U) | Kinase (U) | Ligase (U) | Lane | Pol (U) | Kinase (U) | Ligase (U) | Lane | Pol (U) | Kinase (U) | Ligase (U) |
| 1 | 2.5 | 2.5 | 0.25 U | 51 | 2.5 | 0.1 | 0.25 U | 101 | 2.5 | 0.5 | 0.25 U |
| 2 | 2.5 | 2 | 0.25 U | 52 | 2.5 | 0 | 0.25 U | 102 | 2.5 | 0.25 | 0.25 U |
| 3 | 2 | 2.5 | 0.25 U | 53 | 2 | 0.1 | 0.25 U | 103 | 2 | 0.5 | 0.25 U |
| 4 | 2 | 2 | 0.25 U | 54 | 2 | 0 | 0.25 U | 104 | 2 | 0.25 | 0.25 U |
| 5 | 1.5 | 2.5 | 0.25 U | 55 | 1.5 | 0.1 | 0.25 U | 105 | 1.5 | 0.5 | 0.25 U |
| 6 | 1.5 | 2 | 0.25 U | 56 | 1.5 | 0 | 0.25 U | 106 | 1.5 | 0.25 | 0.25 U |
| 7 | 1 | 2.5 | 0.25 U | 57 | 1 | 0.1 | 0.25 U | 107 | 1 | 0.5 | 0.25 U |
| 8 | 1 | 2 | 0.25 U | 58 | 1 | 0 | 0.25 U | 108 | 1 | 0.25 | 0.25 U |
| 9 | 0.5 | 2.5 | 0.25 U | 59 | 0.5 | 0.1 | 0.25 U | 109 | 0.5 | 0.5 | 0.25 U |
| 10 | 0.5 | 2 | 0.25 U | 60 | 0.5 | 0 | 0.25 U | 110 | 0.5 | 0.25 | 0.25 U |
| 11 | 0.25 | 2.5 | 0.25 U | 61 | 0.25 | 0.1 | 0.25 U | 111 | 0.25 | 0.5 | 0.25 U |
| 12 | 0.25 | 2 | 0.25 U | 62 | 0.25 | 0 | 0.25 U | 112 | 0.25 | 0.25 | 0.25 U |
| 13 | 0.1 | 2.5 | 0.25 U | 63 | 0.1 | 0.1 | 0.25 U | 113 | 0.1 | 0.5 | 0.25 U |
| 14 | 0.1 | 2 | 0.25 U | 64 | 0.1 | 0 | 0.25 U | 114 | 0.1 | 0.25 | 0.25 U |
| 15 | 0 | 2.5 | 0.25 U | 65 | 0 | 0.1 | 0.25 U | 115 | 0 | 0.5 | 0.25 U |
| 16 | 0 | 2 | 0.25 U | 66 | 0 | 0 | 0.25 U | 116 | 0 | 0.25 | 0.25 U |
| 17 | 100 bp ladder | | | 67 | 100 bp ladder | | | 117 | 100 bp ladder (Promega) | | |
| 18 | 2.5 | 1.5 | 0.25 U | 68 | 2.5 | 2.5 | 0.25 U | 118 | 2.5 | 0.1 | 0.25 U |
| 19 | 2.5 | 1 | 0.25 U | 69 | 2.5 | 2 | 0.25 U | 119 | 2.5 | 0 | 0.25 U |
| 20 | 2 | 1.5 | 0.25 U | 70 | 2 | 2.5 | 0.25 U | 120 | 2 | 0.1 | 0.25 U |
| 21 | 2 | 1 | 0.25 U | 71 | 2 | 2 | 0.25 U | 121 | 2 | 0 | 0.25 U |
| 22 | 1.5 | 1.5 | 0.25 U | 72 | 1.5 | 2.5 | 0.25 U | 122 | 1.5 | 0.1 | 0.25 U |
| 23 | 1.5 | 1 | 0.25 U | 73 | 1.5 | 2 | 0.25 U | 123 | 1.5 | 0 | 0.25 U |
| 24 | 1 | 1.5 | 0.25 U | 74 | 1 | 2.5 | 0.25 U | 124 | 1 | 0.1 | 0.25 U |
| 25 | 1 | 1 | 0.25 U | 75 | 1 | 2 | 0.25 U | 125 | 1 | 0 | 0.25 U |
| 26 | 0.5 | 1.5 | 0.25 U | 76 | 0.5 | 2.5 | 0.25 U | 126 | 0.5 | 0.1 | 0.25 U |
| 27 | 0.5 | 1 | 0.25 U | 77 | 0.5 | 2 | 0.25 U | 127 | 0.5 | 0 | 0.25 U |
| 28 | 0.25 | 1.5 | 0.25 U | 78 | 0.25 | 2.5 | 0.25 U | 128 | 0.25 | 0.1 | 0.25 U |
| 29 | 0.25 | 1 | 0.25 U | 79 | 0.25 | 2 | 0.25 U | 129 | 0.25 | 0 | 0.25 U |
| 30 | 0.1 | 1.5 | 0.25 U | 80 | 0.1 | 2.5 | 0.25 U | 130 | 0.1 | 0.1 | 0.25 U |
| 31 | 0.1 | 1 | 0.25 U | 81 | 0.1 | 2 | 0.25 U | 131 | 0.1 | 0 | 0.25 U |
| 32 | 0 | 1.5 | 0.25 U | 82 | 0 | 2.5 | 0.25 U | 132 | 0 | 0.1 | 0.25 U |
| 33 | 0 | 1 | 0.25 U | 83 | 0 | 2 | 0.25 U | 133 | 0 | 0 | 0.25 U |

| | | | | | |
|---|---|---|---|---|---|
| 34 | 100 bp ladder(Promega) | 84 | 100 bp ladder(Promega) | 134 | 100 bp ladder (Promega) |

| | | | | | | | |
|---|---|---|---|---|---|---|---|
| 35 | 2.5 | 0.5 | 0.25 U | 85 | 2.5 | 1.5 | 0.25 U |
| 36 | 2.5 | 0.25 | 0.25 U | 86 | 2.5 | 1 | 0.25 U |
| 37 | 2 | 0.5 | 0.25 U | 87 | 2 | 1.5 | 0.25 U |
| 38 | 2 | 0.25 | 0.25 U | 88 | 2 | 1 | 0.25 U |
| 39 | 1.5 | 0.5 | 0.25 U | 89 | 1.5 | 1.5 | 0.25 U |
| 40 | 1.5 | 0.25 | 0.25 U | 90 | 1.5 | 1 | 0.25 U |
| 41 | 1 | 0.5 | 0.25 U | 91 | 1 | 1.5 | 0.25 U |
| 42 | 1 | 0.25 | 0.25 U | 92 | 1 | 1 | 0.25 U |
| 43 | 0.5 | 0.5 | 0.25 U | 93 | 0.5 | 1.5 | 0.25 U |
| 44 | 0.5 | 0.25 | 0.25 U | 94 | 0.5 | 1 | 0.25 U |
| 45 | 0.25 | 0.5 | 0.25 U | 95 | 0.25 | 1.5 | 0.25 U |
| 46 | 0.25 | 0.25 | 0.25 U | 96 | 0.25 | 1 | 0.25 U |
| 47 | 0.1 | 0.5 | 0.25 U | 97 | 0.1 | 1.5 | 0.25 U |
| 48 | 0.1 | 0.25 | 0.25 U | 98 | 0.1 | 1 | 0.25 U |
| 49 | 0 | 0.5 | 0.25 U | 99 | 0 | 1.5 | 0.25 U |
| 50 | 0 | 0.25 | 0.25 U | 100 | 0 | 1 | 0.25 U |

Samples* in lanes 1-67 were treated at 37 C. 10 min, then 2 hrs at room temp.
Samples* in lanes 68-134 were treated 2 hrs at room temp.
* Excludes 100 bp ladder samples (Lanes 17, 34, 67, 84, 117, 134).

As can be seen from the data in FIG. 6, the Sticky RICE reaction efficiently joined the two DNA molecules over a wide range of polymerase, kinase and ligase levels. The most efficient reactions are in bold in the table above. Also the Sticky RICE reaction works either at a single temp (room temperature only) or when treated first at 37C and then later shifted to room temperature.

### Example 6

### Effects of Temperature and Enzyme Levels on the Sticky RICE Reaction Efficiency

Sticky RICE reactions were assembled as follows: The final volume of each reaction was 12.4 ul. Each reaction contained 1 ul 10×NEB ligase buffer, 25 ng of the 775 bp fragments described in Example 5 and 8 ng of the 225 bp fragment described in Example 5, as well as 1 ul 2 mM dTTP/dATP. Various amounts of polymerase (T4 DNA polymerase, Novagen, LIC qualified), ligase (Gibco) and kinase (NEB T4 polynucleotide kinase) were added to various reactions and the final volume brought to 12.4 ul with dH₂O, as needed.

TABLE 2
Reaction Conditions For Example 6

| Rxn #: | Pol (U) | Kinase (U) | Ligase | 37 C. | RT |
|---|---|---|---|---|---|
| 1 | 1 unit | 2 Units | 0 | 10 min | 60 min |
| 2 | 1 | 2 | 0.1 | 10 min | 60 min |
| 3 | 1 | 2 | 0.25 | 10 min | 60 min |
| 4 | 1 | 2 | 0.5 | 10 min | 60 min |
| 5 | 1 | 2 | 0.75 | 10 min | 60 min |
| 6 | 1 | 2 | 1 | 10 min | 60 min |
| 7 | 1 | 2 | 2 | 10 min | 60 min |
| 8 | 1 | 2 | 0.25 | 10 min | 60 min |
| 9 | 0.25 | 0.5 | 0 | 10 min | 60 min |
| 10 | 0.25 | 0.5 | 0.1 | 10 min | 60 min |
| 11 | 0.25 | 0.5 | 0.25 | 10 min | 60 min |
| 12 | 0.25 | 0.5 | 0.5 | 10 min | 60 min |
| 13 | 0.25 | 0.5 | 0.75 | 10 min | 60 min |
| 14 | 0.25 | 0.5 | 1 | 10 min | 60 min |
| 15 | 0.25 | 0.5 | 2 | 10 min | 60 min |
| 16 | 0.25 | 0.5 | 0.75 | 10 min | 60 min |

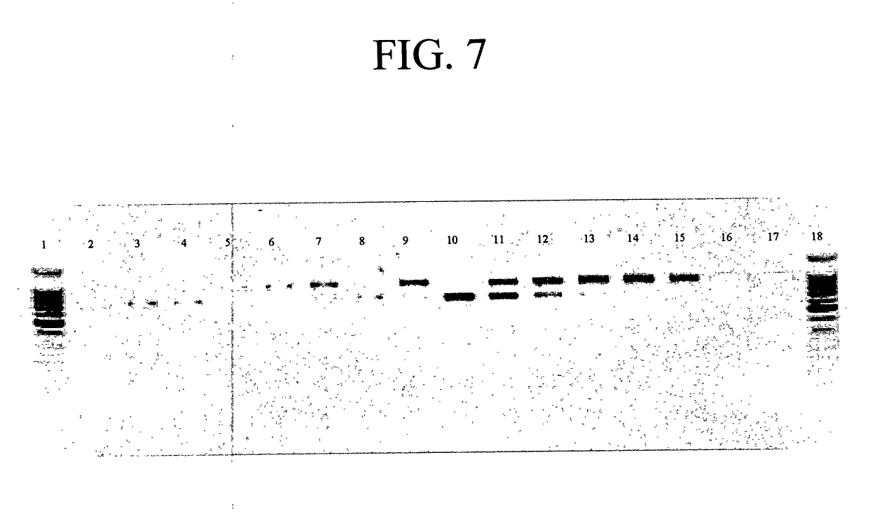

All three enzyme activities were added to all reactions, except for the control reactions (1 and 9), which had no ligase added. Reactions were incubated at 37C for 10 minutes, then left at room temperature for 1 hour with the following exceptions: For reactions 8 and 16 only the polymerase and kinase activities were added, the reaction incubated at 37C for 10 minutes, then incubated at 75C for 10 minutes to inactivate kinase and polymerase. Reactions 8 and 16 were then cooled to room temp. and ligase added at the levels described above. Finally the reactions (8 and 16) were incubated at room temp for 1 hour after the addition of ligase. All reactions were incubated at 75 C for 10 minutes prior to being loaded onto a 1.5% agarose gel in 1×TAE. DNA bands were visualized by staining gel with EtBr and UV as described for Example 1 and is shown in FIG. 7, below. Note that the presence and intensity of the 1 Kb band demonstrates the efficiency of the sticky RICE reaction. The Sticky RICE reaction can be performed by treating the DNAs with kinase and polymerase and then inactivating the polymerase and kinase activities before adding the ligase (reactions 8 and 16).

In FIG. 7, the lanes are numbered left to right. Lane 1: Promega 100 bp ladder. Lanes 2-17, reactions 1-16, respectively. Lane 18, Promega 100 bp ladder.

### Example 7

### Joining of PCR Products: Sticky RICE Cloning System

Directional Cloning System

Directional joining of PCR products using novel sticky RICE technology allows fast and efficient construction of recombinant DNAs from PCR products generated with proofreading or non-proofreading polymerases.

Features and Benefits

Join PCR products in less than 1 hour

Requires less primer design than sequence overlap extension (SOE)

High specificity directional joining of PCR products

Single temperature reaction

No restriction enzymes required

Principle

Sticky RICE, like the PCR-based technique of sequence overlap extension (SOE) can be used to directionally join double stranded DNA molecules without the use of restriction enzymes.

Using the Sticky RICE enzyme mixture, sticky ends are generated, annealed and DNAs joined in a single temperature reaction in less time than an SOE reaction. Since primer design is less complex than with SOE the reaction is both easier and less expensive, and DNAs are joined without the use of error prone DNA polymerases or thermocycling steps.

Specific Recombination in Less Than 1 Hour

Sticky RICE Recombination

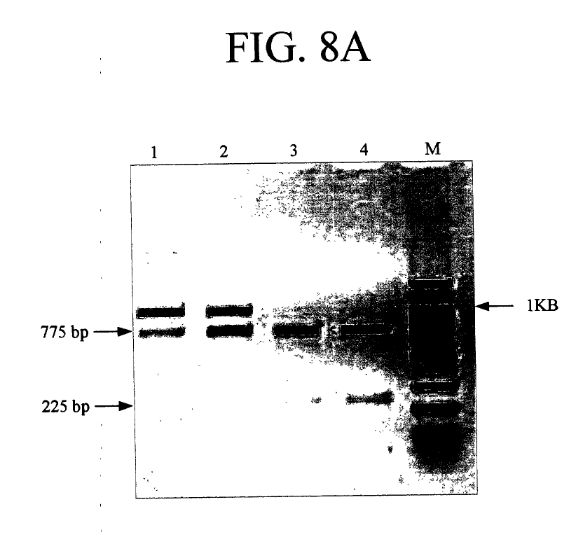

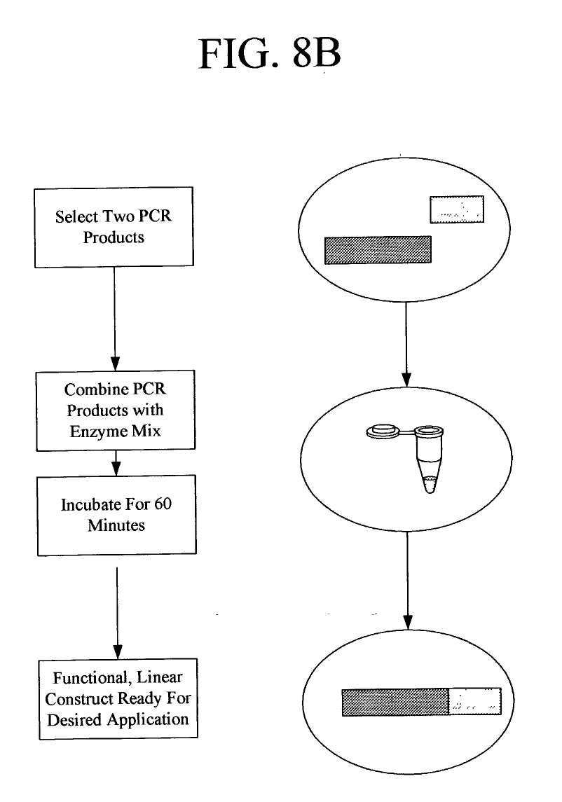

FIG. 8A shows the results of directional joining of a 225 and 725 bp PCR product sharing a 3 bp overlap. PCR products were combined with various amounts of sticky rice enzyme mixture and incubated at room temperature for 1 hour. Reactions were analyzed on a 1.5% agarose gel. Lanes 1-4, sticky RICE reactions with 0.5, 0.25, 0.1 or 0 ul of sticky RICE enzyme mix; M, 100 bp Marker. FIG. 8B depicted the basic steps of Sticky RICE Recombination in accordance with the present invention.

### Example 8

### Cloning of PCR Products: Sticky RICE Cloning System

Directional Cloning System

Directional ligation of PCR products using novel sticky RICE technology allows fast and efficient ligation of PCR products, generated using either proofreading or non-proofreading polymerases, into plasmids. Features and benefit

No restriction enzymes needed

Directional cloning

Clone PCR products of proofreading or non-proofreading polymerases

Rapid ligation time

High cloning efficiency

Principle

Use the Sticky RICE technique to directionally ligate PCR products into a vector of choice without using restriction enzymes. PCR products amplified with any polymerase (proofreading or non-proofreading) can be directly cloned.

Using the sticky RICE enzyme mixture sticky ends are generated, annealed and DNAs joined in a single temperature reaction in less time than a typical restriction enzyme digestion. Recombinant plasmids are generated in a simple room temperature reaction.

High Efficiency Directional Cloning into a 10 kb Plasmid

Sticky RICE Cloning Kit Procedure

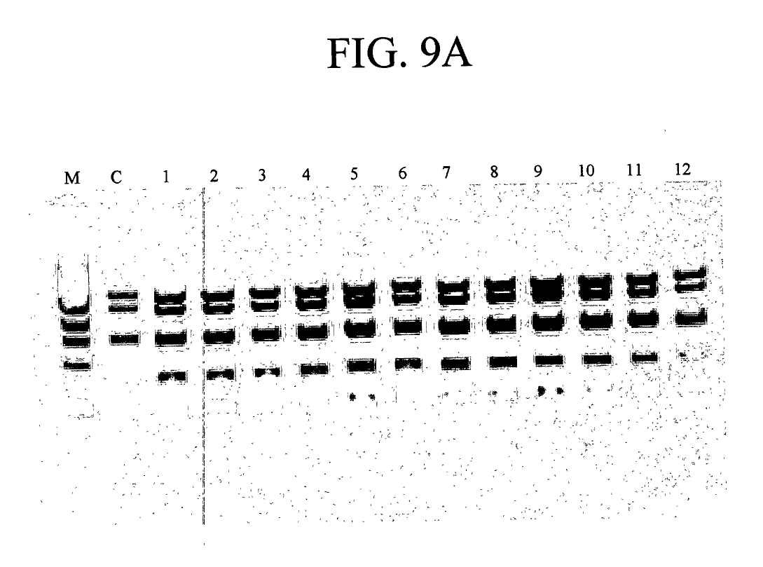

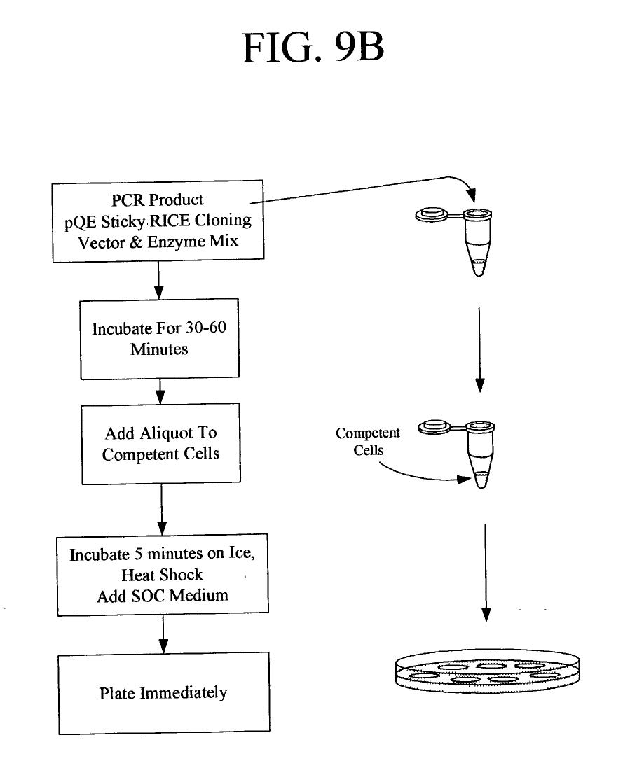

FIG. 9A depicts results of an experiment where a 0.8 kb PCR product was ligated into a 10 kb TMV expression vector plasmid using Sticky RICE. DNA samples from 12 isolates (lanes 1-12) were digested with HindIII and compared to HindIII digested positive control DNA (C). All 12 isolates gave the expected restriction enzyme digestion pattern. Lane M; MW marker. FIG. 9B shows the basic steps of a Sticky RICE Cloning Kit Procedure in accordance with the present invention.

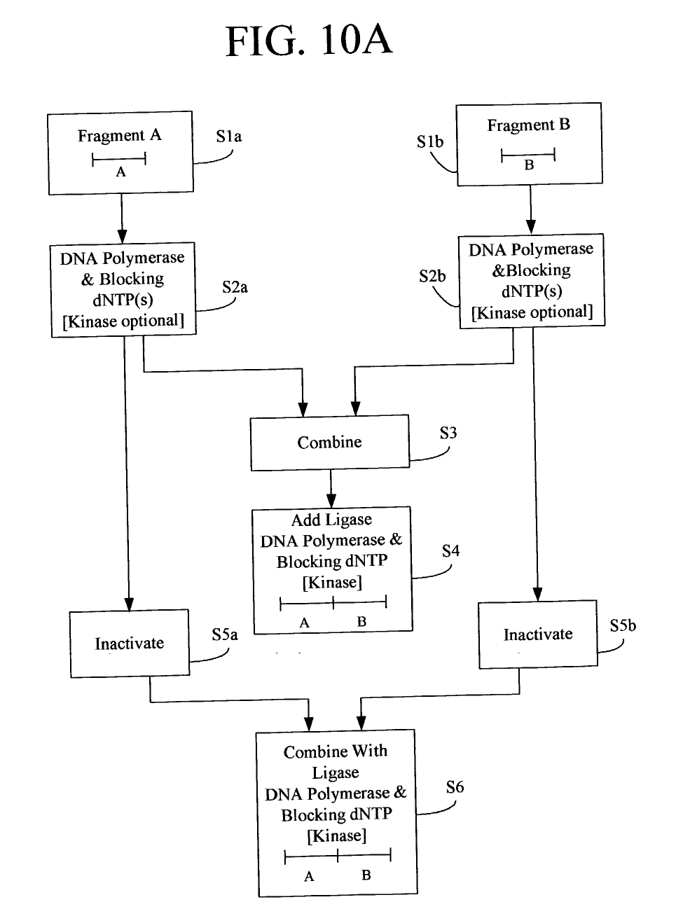

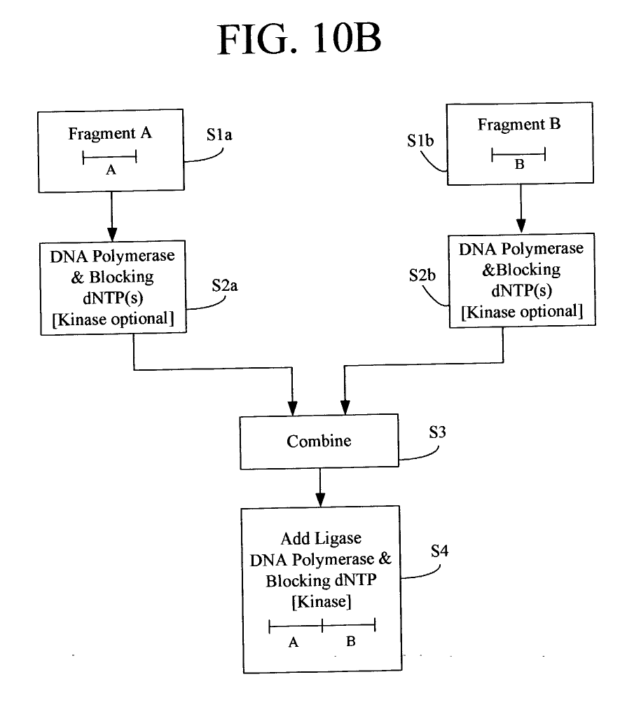

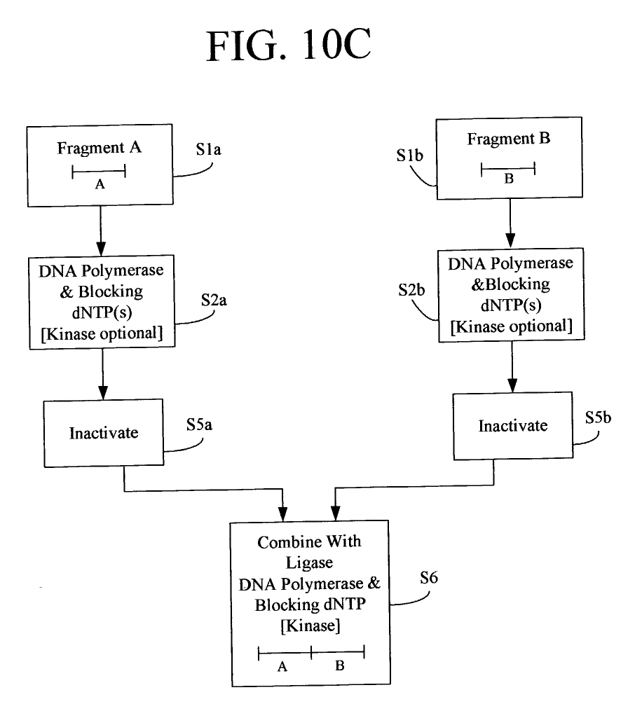

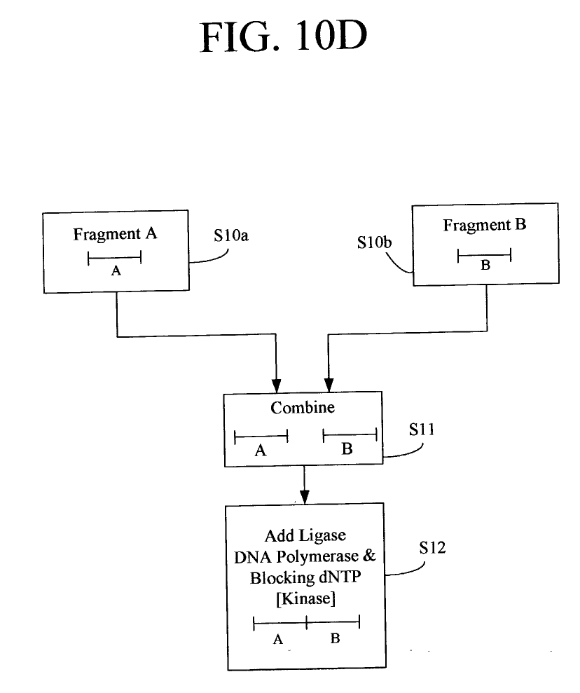

In FIG. 10A, the basic steps of the Sticky RICE methodology are represented showing alternative ways to perform Sticky RICE and thereby joining DNA fragments. Specifically, multiple double stranded DNA fragments A and B, shown in steps S1a and S1b, respectively, in FIG. 10A, are treated with DNA polymerase and the appropriate blocking dNTPs (and optional DNA kinase activity) in separate reactions, as represented at steps S2a and S2b, respectively. In steps S2a and S2b, DNA kinase activity is optional and is therefore shown in parenthesis. Thereafter, there are two alternatives. First, following DNA polymerase treatment in the presence of blocking dNTP(s), the fragments can be directly combined, as indicated at step S3. Next, as shown at step S4, DNA ligase activity, DNA polymerase and blocking dNTP are added to the combined fragments, resulting in the annealing and covalent joining of fragments A and B. The addition of kinase is also optional in step S4

Alternatively, after steps S2a and S2b, as shown in FIG. 10A, the fragments are inactivated at steps S5a and S5b by chemical means (clean up of DNA using DNA purification kits such as Stratagene PCR clean up kit, or Qiaquick DNA clean up kit, etc.) or heat treatment (for example 75° C. for 10 min) of the reactions, or the DNA can be separated from the enzymes (pol and kinase) by physical separation (for example use of a CentriSep spin column, Princeton Separations, will remove unincorporated dNTPs, and proteins, from the reaction). Next, as indicated at step S6, the fragments are combined with ligase, DNA polymerase and blocking dNTP. Use if kinase in step S6 is optional.

To more clearly represent the two embodiments depicted in FIG. 10A, FIGS 10B and 10C are provided with the two alternative embodiments separated. Specifically, in FIG. 10B, multiple double stranded DNA fragments A and B, shown in steps S1a and S1b, respectively in FIG. 10B, are treated with DNA polymerase and the appropriate blocking dNTPs (and optional DNA kinase activity) in separate reactions, as represented at steps S2a and S2b, respectively. In steps S2a and S2b, kinase activity is optional and is therefore shown in parenthesis. Thereafter, the fragments are directly combined, as indicated at step S3. Next, as shown at step S4, DNA ligase activity, DNA polymerase and blocking dNTPs are added to the combined fragments, resulting in the annealing and covalent joining of fragments A and B. The addition of kinase is also optional in step S4

As shown in FIG. 10C, after steps S2a and S2b, the enzymes are inactivated at steps S5a and S5b by chemical means (clean up of DNA using DNA purification kits such as Stratagene PCR clean up kit, or Qiaquick DNA clean up kit, etc.) or heat treatment (for example 75° C. for 10 min) of the reactions, or the DNA can be separated from the enzymes (pol and kinase) by physical separation (for example use of a CentriSep spin column, Princeton Separations, will remove unincorporated dNTPs, and proteins, from the reaction). Next, as indicated at step S6, after the separation of enzymes from the DNA fragments, or inactivation of the enzymes, the fragments are then combined and DNA ligase added to the combined DNAs, allowing the DNA fragments to anneal and become covalently joined. Alternatively, DNA polymerase and additional blocking dNTPs, if needed, and kinase activities can be added in addition to DNA ligase activity at this step.

In another embodiment of the present invention, shown in FIG. 10D, the fragments A and B in steps S10a and S10b, respectively, are combined in a vessel, as indicated at step S11, then DNA polymerase, blocking dNTPs, kinase if needed, and DNA ligase is added to the combined DNAs allowing the DNA fragments to anneal and become covalently joined, as indicated at step S12.

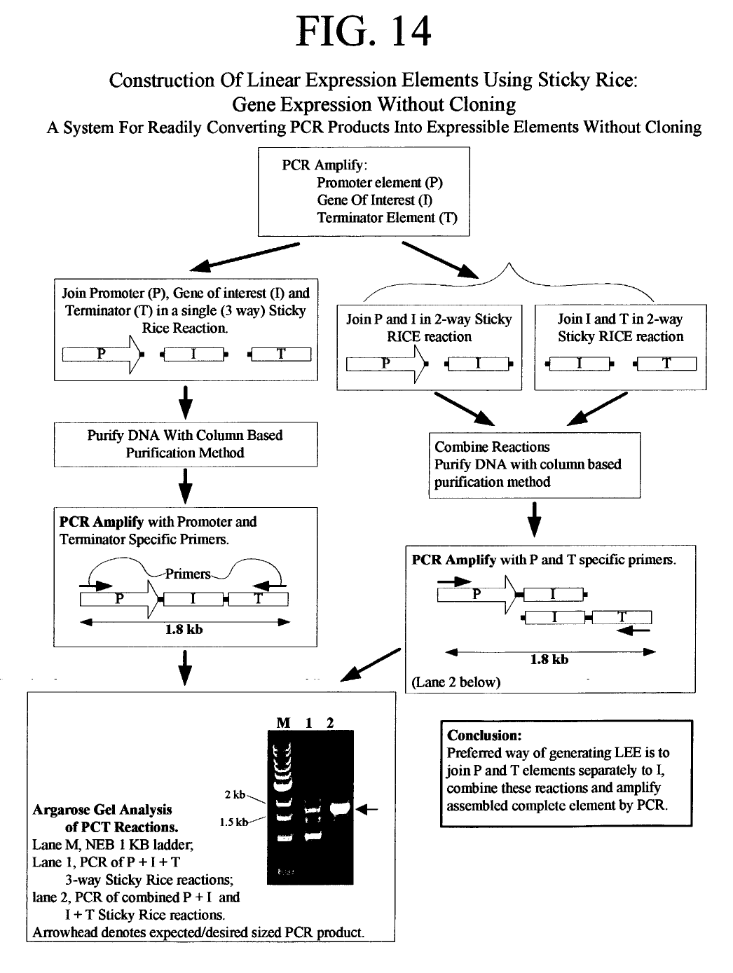

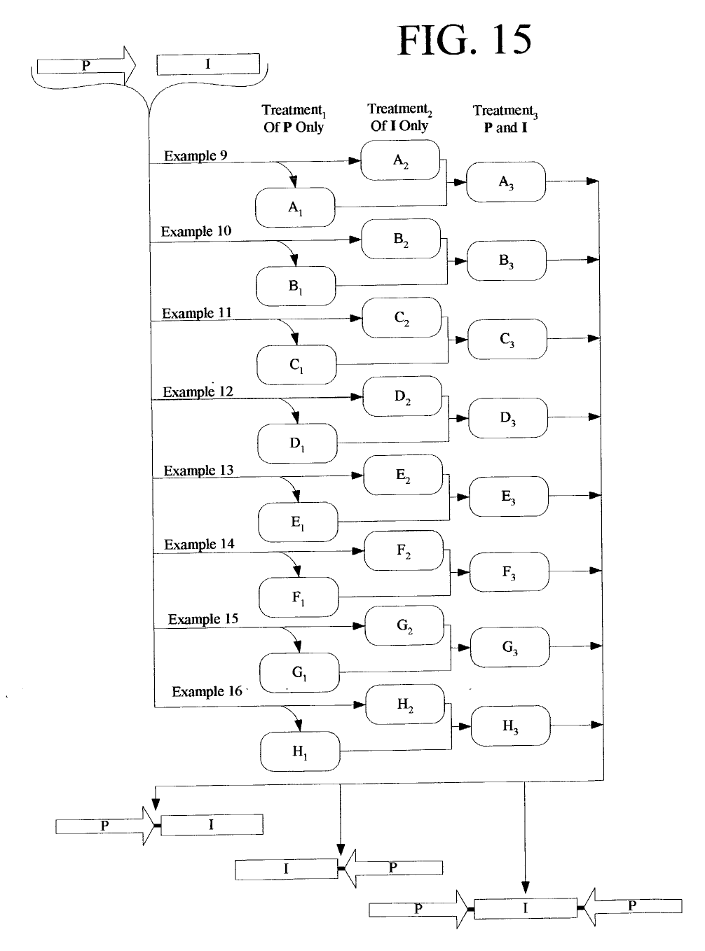

FIG. 14 shows generically a variety of alternative combination of steps for practicing the present invention that are expanded on and further explained with respect to FIGS. 15, 16A, 16B, 16C and 17. Specifically, FIG. 14 shows two alternative processing paths for the construction of linear expression elements, and more specifically gene expression without cloning using the methods of the present invention, that are herein referred to as the Sticky RICE methodology. As shown in FIG. 14, a promoter element P, a gene of interest I and a terminator element T are PCR amplified. Thereafter, two differing processing paths may be followed, in accordance with different embodiments of the present invention.

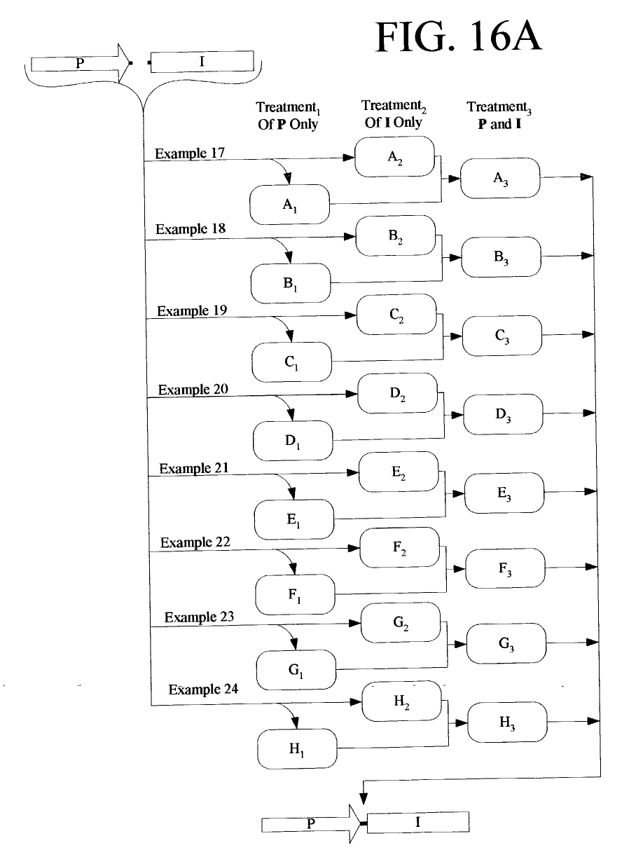

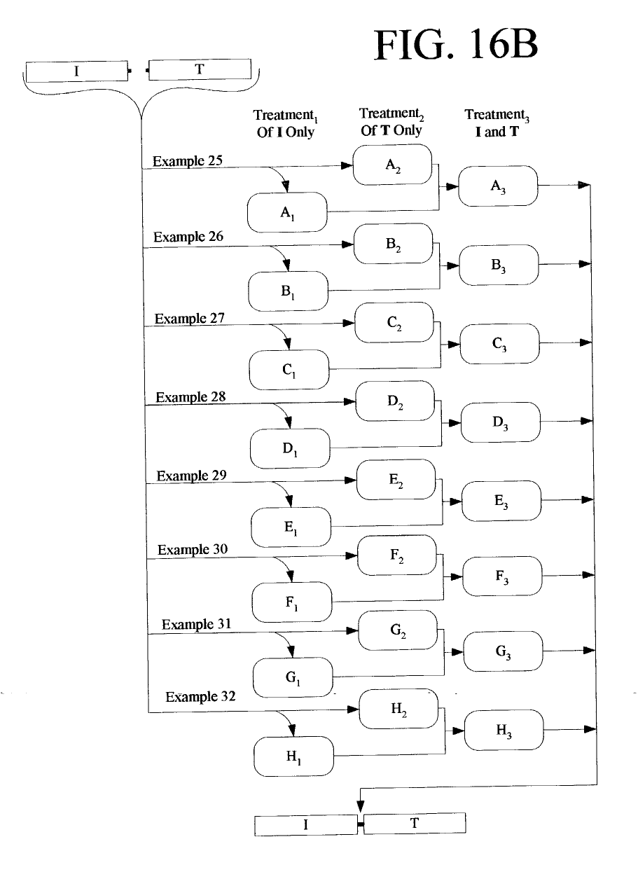

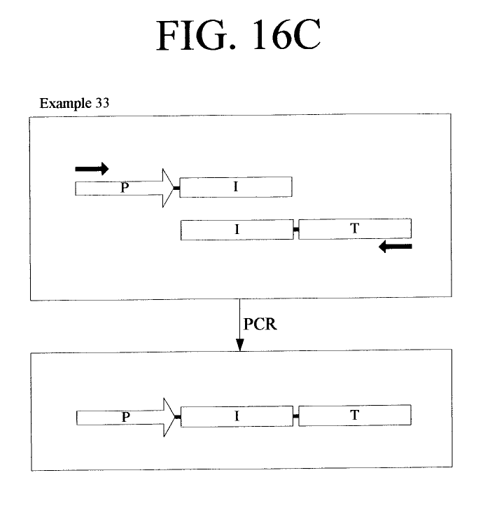

For example, on the right hand side of FIG. 14, the promoter P and a quantity of the gene of interest I may be joined in a 2-way Sticky RICE reaction, and simultaneously another quantity of the gene of interest I may be joined with the terminator T. Thereafter, the reactions are combined and the DNA purified using a column based purification method. Next, the promoter P and terminator T are PCR amplified using specific primers resulting in a desired construct that includes the promoter P, the gene of interest I and the terminator T. Various combinations of treatments represented on the right hand side of FIG. 14 are further embellished and described with respect to FIGS. 15, 16A, 16B and 16C and Examples 9-33.

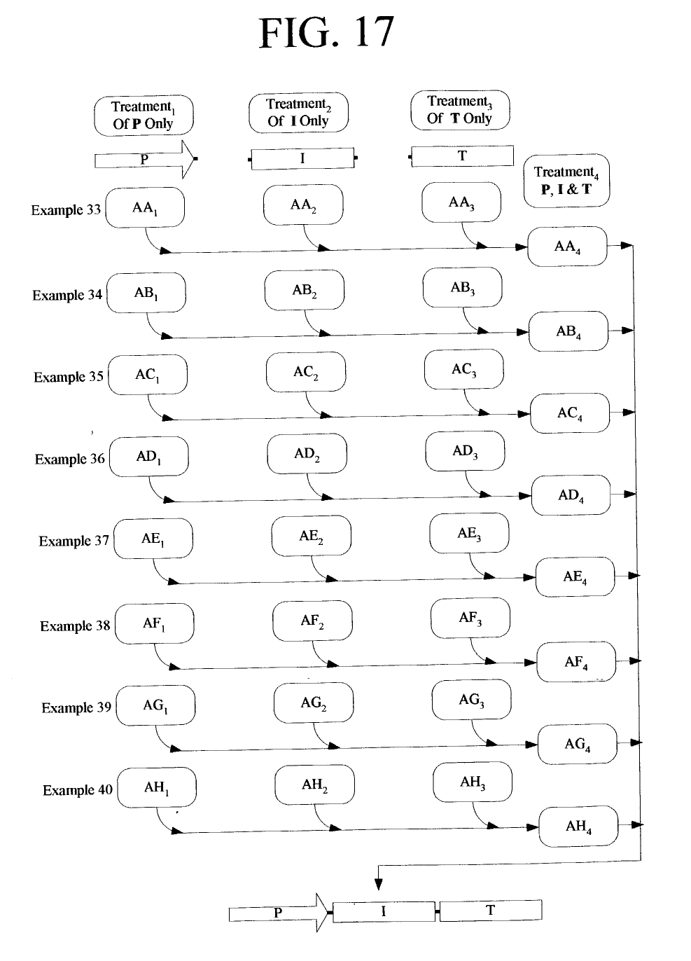

On the left hand side of FIG. 14, the promoter P, the gene of interest I and the terminator T are combined in a single 3 way reaction. The resulting DNA is then purified using column based purification methods and then PCR amplified with promoter and terminator specific primers. Various combinations of treatments represented on the left hand side of FIG. 14 are further embellished and explained with respect to FIG. 17 and Examples 34-41.

The plurality of embodiments presented below include various combinations of reactions where the individual reactions have already been described above. Therefore, Embodiments 9-41 are presented below in a concise tabular manner to more clearly demonstrate the variations in the combinations of treatments used to practice the present invention, along with comments on the various combinations of treatments.

FIGS. 15, 16A, 16B, 16C and 17 show a variety of combination of steps that are used to practice the present invention. The basic steps and embodiments represented in FIGS. 15, 16A, 16B and 16C are tallied in the tables below:

| Step | Treatment |
|---|---|
| A1 | Kinase treatment |
| A2 | Phosphatase/no treatment |
| A3 | Polymerase + blocking dNTPs and ligase (optional kinase) |
| B1 | Phosphatase/no treatment |
| B2 | Kinase Treat |
| B3 | Polymerase + blocking dNTPs and ligase (optional kinase) |
| C1 | Phosphatase/no treatment |
| C2 | Phosphatase/no treatment |
| C3 | Polymerase + blocking dNTPs and ligase and kinase |
| D1 | Kinase and Polymerase + blocking dNTPs treat |
| D2 | Kinase and Polymerase + blocking dNTPs treat |
| D3 | Ligase (Optional polymerase and blocking dNTPs, optional |
| | kinase) |
| E1 | Kinase and Polymerase + blocking dNTPs treat |
| E2 | Polymerase + blocking dNTPs treat |
| E3 | Ligase (optional polymerase and blocking dNTPs, optional |
| | kinase) |
| F1 | Polymerase + blocking dNTPs treat |
| F2 | Kinase and Polymerase + blocking dNTPs treat |
| F3 | Ligase (optional polymerase and blocking dNTPs, optional |
| | kinase) |
| G1 | Polymerase and blocking dNTPs treatement |
| G2 | Polymerase and blocking dNTPs treatement |
| G3 | Kinase and ligase treatment (optional polymerase and |
| | blocking dNTPS |
| H1 | Kinase treatment |
| H2 | Kinase treatment |
| H3 | Polymerase and blocking dNTPs, ligase, (optional kinase) |

| Figure | Embodiment | Steps | Treatment Of: |
|---|---|---|---|
| 15 | 9 | A₁, A₂ & A₃ | P, I and P & I respectively |
| 15 | 10 | B₁, B₂ & B₃ | P, I and P & I respectively |
| 15 | 11 | C₁, C₂ & C₃ | P, I and P & I respectively |
| 15 | 12 | D₁, D₂ & D₃ | P, I and P & I respectively |
| 15 | 13 | E₁, E₂ & E₃ | P, I and P & I respectively |
| 15 | 14 | F₁, F₂ & F₃ | P, I and P & I respectively |
| 15 | 15 | G₁, G₂ & G₃ | P, I and P & I respectively |
| 15 | 16 | H₁, H₂ & H₃ | P, I and P & I respectively |
| 16A | 17 | A₁, A₂ & A₃ | P, I and P & I respectively |
| 16A | 18 | B₁, B₂ & B₃ | P, I and P & I respectively |
| 16A | 19 | C₁, C₂ & C₃ | P, I and P & I respectively |
| 16A | 20 | D₁, D₂ & D₃ | P, I and P & I respectively |
| 16A | 21 | E₁, E₂ & E₃ | P, I and P & I respectively |
| 16A | 22 | F₁, F₂ & F₃ | P, I and P & I respectively |
| 16A | 23 | G₁, G₂ & G₃ | P, I and P & I respectively |
| 16A | 24 | H₁, H₂ & H₃ | P, I and P & I respectively |
| 16B | 25 | A₁, A₂ & A₃ | I, T and I & T respectively |
| 16B | 26 | B₁, B₂ & B₃ | I, T and I & T respectively |
| 16B | 27 | C₁, C₂ & C₃ | I, T and I & T respectively |
| 16B | 28 | D₁, D₂ & D₃ | I, T and I & T respectively |
| 16B | 29 | E₁, E₂ & B₃ | I, T and I & T respectively |
| 16B | 30 | F₁, F₂ & F₃ | I, T and I & T respectively |
| 16B | 31 | G₁, G₂ & G₃ | I, T and I & T respectively |
| 16B | 32 | H₁, H₂ & H₃ | I, T and I & T respectively |
| 16C | 33 | PCR | P & I and I & T |

Comments on Treatments:

Sticky RICE joining of DNA molecules is accomplished through the use of multiple enzymatic activities, either in a concurrent or step-wise fashion. Examples of how the individual or co-enzymatic treatments can be accomplished are described below. Permutations of the described treatments can also be performed to accomplish the desired treatment outcome.

Phosphatase Treatment/No Treatment

If one wishes to remove 5′ phosphate groups from a DNA (as in FIG. 15A₂) this can be accomplished by incubating a DNA molecule in the presence of shrimp alkaline phosphatase (Boehringer-mannheim) or Calf alkaline phosphatase (New England Biolabs) according to the manufacturers instructions (of buffer, time, temperature, DNA concentration, etc) using either the buffer supplied by the manufacturer or other suitable buffer/reaction conditions which allow the enzyme to effectively remove 5′ phosphates from DNA.

If desired the phosphatase treatement can be terminated by high temperature incubation of the reaction (for example shrimp alkaline phosphatase is inactivated by 15 min treatment at 65° C.), or purifying the DNA using a commercial DNA clean up kit according to the manufacturers instructions.

If the DNA does not have 5′ phosphate groups (because of a previous treatment with phosphatase, or the DNA fragment was amplified using PCR and primers without 5′ phosphate groups, for example), the phosphatase treatment is not necessary.

The phosphatase/No treatment applies to steps (A2, B1, C1, C2, AA1, AA2, AA3, AB1, AB3, AC2 in FIGS. 15-17)

Kinase Treatment:

If one wishes to add, or ensure that a DNA molecule has 5′ phosphate groups, this can be accomplished by incubating a DNA molecule in the presence of rATP and a nucleotide kinasing enzyme such as polynucleotide kinase which is available from a number of sources (inlcuding New England Biolabs, Promega, Invitrogen, etc.), according to the manufacturers instructions of buffer, time, temperature, DNA concentration, etc.

If desired the phosphatase treatement can be terminated by high temperature incubation of the reaction (for example T4 polynucleotide kinase from New England Biolabs is inactivated by treatement at 65C for 20 minutes), or by purifying the DNA using a commercial DNA clean up kit according to the manufacturers instructions.

The Kinase treatment step applies to steps (A1, B2, H1, H2, AB2, AC1, AC3, AH1, AH2, AH3 in FIGS. 15-17)

Polymerase Treatment with Blocking NTPs

If one wishes to generate 5′ overhangs on a DNA molecule, this can be accomplished by incubating a DNA molecule in the presence of a DNA polymerase activity with 3′ to 5′ exonuclease activity using the manufacturers instructions as a guideline. For example T4 DNA polymerase from New England Biolabs can function under a variety of temperature and buffer conditions and the concentration of blocking dNTPs to be used are suggested to be 100 mM.

If desired the polymerase+blocking dNTP reaction can be terminated by high temperature incubation of the reaction (for example T4 DNA polymerase from New England Biolabs is inactivated by treatment at 75° C. for 10 minutes), or by purifying the DNA using a commercial DNA clean up kit according to the manufacturers instructions.

The polymerase with blocking dNTP(s) step applies to steps (E2, F1, G1, G2, AD2, AE1, AE2, AE3, AG1, AG3 in FIGS. 15-17).

Polymerase+Blocking dNTP(s) and Kinase Co-Treatment.

If one desires to ensure that the 5′ ends of a DNA molecule contain 5′ phosphate groups and also to generate or maintain 5′ overhangs on a DNA molecule during the same step a DNA molecule can be incubated in the presence of DNA polymerase (with 3′ to 5′ exonuclease activity and the appropriate blocking dNTPs) and a polynucleotide kinase activity in the presence of rATP (manufacturers of polynucleotide kinase generally suggest that the final rATP concentration by 1 mM) in a buffer in which both enzyme activities are active. For example both T4 DNA polymerase and T4 polynucleotide kinase have been demonstrated to function in T4 DNA ligase buffer (New England Biolabs) supplemented with the appropriate blocking dNTPs. (FIG. 6).

If desired the polymerase+blocking dNTP and coupled kinase treatement reaction can be terminated by high temperature incubation of the reaction (for example 75° C. for 20 minutes would inactivate both T4 DNA polymerase and T4 polynucleotide kinase activities), or by purifying the DNA using a commercial DNA clean up kit according to the manufacturers instructions.

The polymerase+blocking dNTP(s) and kinase co-treatment applies to steps (D1, D2, E1, F2, AD1, AD3, AF1, AF2, AF3, AG2 in FIGS. 15-17).

Polymerase+Blocking dNTP(s) and DNA Ligase Co-Treatment.

If one desires to generate or maintain 5′ overhangs on a DNA molecule and also allow complementary 5′ overhangs to anneal and become ligated in a single reaction, this can be accomplished by treating multiple DNA molecules in the presence of a DNA ligase activity and a DNA polymerase activity (with 3′ to 5′ exonuclease activity) in the presence of the appropriate blocking dNTPs.

For example T4 DNA polymerase and T4 DNA ligase from New England Biolabs are both active in 1×T4 DNA ligase buffer (New England Biolabs) supplemented to blocking dNTPs.

If desired the polymerase+blocking dNTP and coupled ligase treatement reaction can be terminated by high temperature incubation of the reaction (for example 75° C. for 20 minutes would inactivate both T4 DNA polymerase and T4 DNA ligase activities), or by purifying the DNA using a commercial DNA clean up kit according to the manufacturers instructions.

The polymerase+blocking dNTP(s) and DNA ligase co-treatment (with or without the optional components described) applies to steps (A3, B3, C3, H3, AA4, AB4, AC4, AH4) in FIGS. 15-17.

Kinase and Ligase Co-Treatment of DNAs.

If one desires to ensure that DNA molecules have 5′ phosphate groups and that DNA fragments can be ligated together in a single step, this can be accomplished by treating multiple DNA molecules in the presence of polynucleotide kinase and DNA ligase activities under suitable reaction conditions. For example T4 polynucleotide kinase and T4 DNA ligase from New England Biolabs are both functional in 1×T4 DNA ligase buffer from New England Biolabs, at a variety of temperatures.

If desired the kinase and coupled ligase treatment reaction can be terminated by high temperature incubation of the reaction (for example 75° C. for 20 minutes would inactivate both T4 DNA polymerase and T4 DNA ligase activities), or by purifying the DNA using a commercial DNA clean up kit according to the manufacturers instructions.

The kinase and ligase co-treatment applies to steps G3, AE4, in FIGS. 15-17. Ligase Treatment

If one wishes to covalently join DNA molecules together this can be accomplished by treating multiple DNAs in the presence of a DNA ligase activity under the appropriate conditions. See manufacturers suggested reaction conditions of buffer, DNA concentration, reaction temperature, etc. Optionally appropriate amounts of kinase and or polymerase in the presence of blocking dNTPs can be also added to the ligase treatment.

The ligase (with optional kinase and or optional polymerase+blocking dNTPs) treatment applies to steps D3, E3, F3, AD4, AF4, AG4) in FIGS. 15-17.

Polymerase+Blocking dNTPs, and Ligase and Kinse Co-Treatment:

If one wishes to maintain or generate 5′ single stranded overhangs on multiple DNA molecules, generate 5′ phosphate groups on DNA molecules, and allow multiple DNA molecules to be joined, this can be accomplished by incubating multiple DNAs in the presence of polymerase activity (+the appropriate blocking dNTPs), DNA kinase activity and DNA ligase activity in a single reaction, using the buffer and enzyme ratios described in FIG. 6, for example.

The Polymerase+blocking dNTPs, and ligase and Kinse co-treatment step applies to steps (C3 and AA4) in FIGS. 15-17.

As presented in FIG. 6, it is important to note that not all combinations of polymerase, kinase and ligase activities can function to generate recombinant DNA molecules. The ratio of the multiple enzyme activities must be balanced to ensure that all enzyme activities function at a rate consistent with the desired output in terms of quality and quantity of recombinant DNA produced in the desired time frame. The in vitro DNA joining assay in FIG. 6 demonstrates that “too much” or “too little” of one or more enzyme activities can dramatically effect the DNA joining reaction rate. Using the assay and conditions similar or identical to those described for FIG. 6 a researcher can readily identify the enzyme mixture(s) which can accomplish the desired output.

Multiple Applications for the Sticky RICE Workflow in FIG. 15.

Using the various combinations of work flow outlined in FIG. 15, sticky RICE can be used to generate recombinant DNAs for a variety of applications including, but not limited to, manual or automated cloning of DNAs into plasmids, production of recombinant RNAs for in vitro or in vivo transcription and/or translation and production of double stranded RNA for RNAi applications.

Cloning Applications:

The work flow outlined in FIG. 15 can also be used for cloning an insert DNA fragment (I) into a plasmid (P) DNA fragment. In this scenario the desired outcome is a circular recombinant DNA molecule which can replicate in a cell such as bacterial, plant, animal, fungal or insect cells, for example. A circular molecule will result from the work flow outlined in FIG. 15, if both ends of the DNA fragment P become ligated to opposite ends of fragment I. This can be readily accomplished by appropriately designing the nucleotide sequence on the ends of fragments P and I so that a circular molecule will result during sticky RICE joining of fragments P and I.

The work flow in FIG. 15 is designed to be modular in nature, to allow the user to treat fragments P and I independently, if desired. This also allows the user to control the treatments of fragments P and I separately, if desired by altering the amount of time, enzyme amounts, etc. in the separate treatments. Enzymatic treatments of the DNAs can be stopped by inactivation of enzymes (by heat treatment, for example) or by separation of the DNA from the enzymes (by purification of DNA by precipitation, or use of commercial DNA “clean up kits” such as Qiagen PCR clean up kits, Stratagene PCR clean up kits, etc.).

In an automated cloning system, for example, it would be possible to pre-treat multiple aliquots (in a 96 well plate, for example) of plasmid vector (fragment P) with a phosphatase enzyme (such as calf alkaline or shrimp alkaline phosphatase) to remove phosphates from the 5′ ends of DNA P (FIG. 15, I1). After adequate phosphatase treatment the phosphatase enzyme can be inactivated by incubating the plate at an elevated temperature. For example shrimp alkaline phosphatase is inactivated by a heat treatment for 15 minutes at 65 C, Boehringer-mannheim. The present invention may be automated using, for instance, a computer controlled liquid handler, such as that marketed and sold by TECAN, Zurich Switzerland.

In a separate plate, aliqouts of various insert DNAs (the I fragments in FIG. 15 workflow) can be pre-treated with a kinase enzyme for, example (FIG. 15, I2). After adequate kinase treatment of a plate of I fragments, the kinase reaction can be terminated by heat inactivation. For example T4 polynucleotide kinase is inactivated by 20 minute treatment at 65 C (New England Biolabs).

The pretreated P and I samples can then be combined (FIG. 1513) and treated with DNA ligase, DNA polymerase+the appropriate blocking dNTPs to allow for the joining of fragments P and I.

In a similar manner, each of the other work flows outlined in FIG. 15 can be readily adapted to an automated cloning system.

Using the guidelines described earlier in this patent, it is possible to design plamsid vector DNAs (P fragments in FIG. 15) and insert DNAs (I fragments in FIG. 15) which can be joined in either a directional or a non-directional orientation via sticky RICE techniques. RNAi/dsRNA applications:

The work flows outlined in FIG. 15 can also be used to generate linear recombinant DNAs which can be useful for generating RNAs.

For example, if one is interested in generating ds RNA molecules (for RNAi applications, for example) the work flow outlined in FIG. 15 can be applied as follows. If fragment I is designed to have identical 5′ single stranded overhangs on each end, and fragment P has complementary 5′ single stranded overhangs, then fragment P can be joined to either or both ends of fragment I to generate the three different linear recombinant DNA molecules represented at the bottom of FIG. 15.

This can be of interest, in particular, if the researcher is interested in generating double stranded RNA molecules in vitro or in vivo. For example, if fragment P contains a functional promoter sequence then transcription of the resulting population of recombinant DNAs with functional promoters attached to the “Left” end only and “Right” end only of fragment I will generate RNA molecules of complementary sequence. These RNA molecules may hybridize and form ds RNA. Similarly transcription of a recombinant DNA with functional promoters on both ends will also result in complementary RNA molecules, which may hybridize to form ds RNA.

If the researcher is interested in generating RNAs which will form hairpins, then fragment I must have a nucleotide sequence which upon transcription will generate an RNA molecule with self complementarity to form a hairpin. One way in which this can be accomplished is by synthesizing an oligonucleotide which is capable of readily forming a primer dimer. For example the oligo nucleotide of the sequence:

5′ CT TAC gTA CTT gCA CCA TCC ATg gA (Seq ID No. 48)

has 8 nt of self complementarity on its 3′ end. Because of this tendency to dimerize at its 3′end (see below):

| | |
|---|---|
| 5′CT TAC gTA CTT gCA CCA TCC ATg gA | (Seq ID No.: 49) |
| \overline{ AGG TAC CT ACc ACG TTC ATG CAT TC5′} | (Seq ID No.: 50) |

when this oligo is allowed to anneal to itself (the estimated Tm of the 8 nts which are complementary would be about 24° C.) in the presence of the appropriate buffer, dNTPs and DNA polymerase enzyme (such as T7-, T4-, or E. coli/Klenow DNA polymerase) a ds inverted repeat DNA (of the following sequence) will be generated:

| | |
|---|---|
| 5′CT TAC gTA CTT gCA CCA TCC ATg gA tgg tgc aag tac gta ag | (Seq ID No.: 51) |
| ga atg cat gaa cgt ggt agg TAC CT ACc ACG TTC ATG CAT TC5′ | (Seq ID No.: 52) |

Note that the critical feature in this example is the self complementarity at the 3′ end of the oligonucleotide and not the oligonucleotide sequence per se; the sequence of the oligonucleotide can be changed. The generated inverted repeat DNA molecule, if transcribed, would generate a double stranded RNA molecule, which could be used for applications such as RNAi for example. To join a DNA promoter element via sticky RICE to one or both ends of the inverted repeat DNA one may treat the inverted repeat DNA sequence with polymerase with 3′ to 5′ exonuclease activity in the presence of dATP and dTTP blocking nucleotides to generate single 5′ nt C overhangs on both ends of the inverted repeat DNA. This can then be joined to a DNA molecule (such as a promoter, for example) with a complementary 5′ G overhang. This joining process can be accomplished using sticky RICE and any of the work flows outlined in FIG. 15.

Because the promoter element can go on either or both ends of the inverted repeat DNA sequence the possible DNA products could be:

P---I
I---P
P---I---P

as represented at the bottom of FIG. 15.

All of these recombinant DNAs (or the appropriate PCR products thereof) can be transcribed (either in vitro and/or in vivo, depending upon the functionality of the promoter in DNA fragment P) to generate ds (hairpin) RNA molecules useful in applications such as RNAi.

Applications for the Work Flow of FIGS. 16A-16C:

The work flows outlined in FIGS. 16A and B can be used to construct primarily a single recombinant DNA molecule. For example if fragments P and I (FIG. 16A) are designed (using the guidelines described earlier in this patent) for sticky RICE joining in a directional orientation, the primary recombinant molecule produced would be the linear recombinants DNA pictured at the bottom of FIG. 16A

If fragment P has promoter activity (either in vitro and/or in vivo) the resulting linear recombinant (or the appropriate PCR product thereof) can be transcribed under the appropriate conditions to generate an RNA molecule.

The recombinant “P—I” molecule can be used for many applications by itself, including in vitro transcription and/or translation applications for producing proteins in vitro or in vivo, for example. The recombinant “P—I” molecule can be used in applications directly or can be amplified up using the polymerase chain reaction and suitable primers. The amplified recombinant molecule can then be used in applications as desired.

In a separate set of reactions, a second fragment “T” (see FIG. 16B) can be designed to be joined to only the “right” end of the same fragment I as in FIG. 16A, to produce the linear recombinant molecule represented in the bottom of FIG. 16B.

If fragment T has promoter activity (either in vitro and/or in vivo) the resulting linear recombinant (or the appropriate PCR product thereof) can be transcribed under the appropriate conditions to generate an RNA molecule.

When the transcripts from the recombinant DNA produced from the work flow in FIG. 16A are in the presence of transcripts from the recombinant DNA produced from the work flow in FIG. 16B, the two populations of RNA molecules may hybridize (because they are complementary in sequence) to generate a ds RNA molecule. This ds RNA generated can be useful for applications such as RNAi for example.

The product of FIG. 16A and FIG. 16B can be transcribed in the same tube, vessel or cell, or in seperate tubes and then combined to generate ds RNAs.

Similar to the description for FIG. 15, the work flows in FIGS. 16A and 16B can also be automated. Fragments P and I and T, for example can be seperately treated in 96 well plates for example and then combined as desired to generate the desired recombinant molecules.

Other applications of the workflows outlined in FIGS. 16A and 16B include the production of gene fusions by joining of multiple DNA fragments. For example a recombinant DNA product of two reading frames encoded by fragments P and I can be joined using the work flow oultined in FIG. 16A or 16B.

For some applications it may be of interest to generate a recombinant DNA molecule of three or more DNAs joined in a directional manner. This can be accomplished using the work flows outlined in FIGS. 16A-16C. For example the output from FIG. 16C is a recombinant DNA of DNAs P, I and T, joined to generate a recombinant of the following orientation “P-I-T” as represented in FIG. 16C. Applications of such a recombinant molecule include in vitro transcription, in vitro transcription and translation and in vivo transcription/and or translation applications.

For example, if one desires to construct a linear recombinant DNA which can be transcribed in vitro or in vivo, it may be of interest to attach a promoter P fragment to one specific end of DNA fragment I and a DNA fragment T, which has transcription termination/polyadenylation functions, etc. to the other end of fragment I. This can be accomplished in a step wise fashion following the work flows outlined in FIGS. 16A-C. To construct the 3-way recombinant DNA molecule described in FIG. 16C, the product of the work flow of FIG. 16A (P-I recombinant) and the product of the work flow from FIG. 16B (the I-T recombinant) can be combined in a single reaction vessel and subjected to polymerase chain reaction (PCR) conditions using primers that anneal specifically to fragments P and T, as represented by the arrows in FIG. 16C. During the polymerase chain reaction the P-I-T recombinant DNA molecule will be generated.

The P-I-T recombinant molecule produced can be used in applications such as protein production, if fragment P has promoter activity, and fragments I and or T contain an open reading frame. The P-I-T recombinant DNA can be used to program in vitro transcription and or transcription/translation reactions to product recombinant protein(s). Similarly the P-I-T recombinant molecule can be introduced into living cells by microinjection, transfection, etc. to allow the P-I-T recombinant to be transcribed in vivo and the RNA potentially translated to produce protein(s) in living cells.

In FIG. 17, various combinations of treatments are depicted in the workflows. The table below sets forth the various steps and the portion of the sequence being treated.

Table Of Various Steps Shown In FIG. 17

| Step | Treatment |
|---|---|
| AA1 | Phosphatase/no treatment |
| AA2 | Phosphatase/no treatment |
| AA3 | Phosphatase/no treatment |
| AA4 | Polymerase + blocking dNTPs and ligase and kinase treat |
| AB1 | Phosphatase/no treatment |
| AB2 | Kinase Treat |
| AB3 | Phosphatase/no treatment |
| AB4 | Polymerase + blocking dNTPs and ligase (optional kinase) |
| AC1 | Kinase treat |
| AC2 | Phosphatase/no treatment |
| AC3 | Kinase treat |
| AC4 | Polymerase + blocking dNTPs and ligase (optional kinase) |
| AD1 | Kinase and Polymerase + blocking dNTPs treat |
| AD2 | Polymerase + blocking dNTPs treat |
| AD3 | Kinase and Polymerase + blocking dNTPs treat |
| AD4 | Ligase (Optional polymerase and blocking dNTPs, optional kinase) |
| AE1 | Polymerase + blocking dNTPs treat |
| AE2 | Polymerase + blocking dNTPs treat |
| AE3 | Polymerase + blocking dNTPs treat |
| AE4 | Ligase and Kinase (optional polymerase and blocking |
| | dNTPs) |
| AF1 | Kinase and Polymerase + blocking dNTPs treat |
| AF2 | Kinase and Polymerase + blocking dNTPs treat |
| AF3 | Kinase and Polymerase + blocking dNTPs treat |
| AF4 | Ligase (optional kinase, optional polymerase and blocking |
| | dNTPs) |
| AG1 | Polymerase + blocking dNTPs treatement |
| AG2 | Kinase and Polymerase + blocking dNTPs treatement |
| AG3 | Polymerase + blocking dNTPs treatement |
| AG4 | Ligase treatment (optional polymerase + blocking dNTPS, |
| | optional kinase) |
| AH1 | Kinase treatment |
| AH2 | Kinase treatment |
| AH3 | Kinase treatment |
| AH4 | Polymerase and blocking dNTPs, ligase, (optional kinase) |

| Figure | Example | Steps | Treatment Of: |
|---|---|---|---|
| 17 | 34 | AA₁, AA₂, AA₃ & AA₄ | P, I, T and P, I & T |
| | | | respectively |
| 17 | 35 | AB₁, AB₂, AB₃ & AB₄ | P, I, T and P, I & T |
| | | | respectively |
| 17 | 36 | AC₁, AC₂, AC₃ & AC₄ | P, I, T and P, I & T |
| | | | respectively |
| 17 | 37 | AD₁, AD₂, AD₃ & AD₄ | P, I, T and P, I & T |
| | | | respectively |
| 17 | 38 | AE₁, AE₂, AE₃ & AE₄ | P, I, T and P, I & T |
| | | | respectively |
| 17 | 39 | AF₁, AF₂, AF₃ & AF₄ | P, I, T and P, I & T |
| | | | respectively |
| 17 | 40 | AG₁, AG₂, AG₃ & AG₄ | P, I, T and P, I & T |
| | | | respectively |
| 17 | 41 | AH₁, AH₂, AH₃ & AH₄ | P, I, T and P, I & T |

The various individual treatment steps outlined in workflows in FIG. 17 and referred to in the tables have all been described above. To avoid duplicative text, comments will not be repeated.

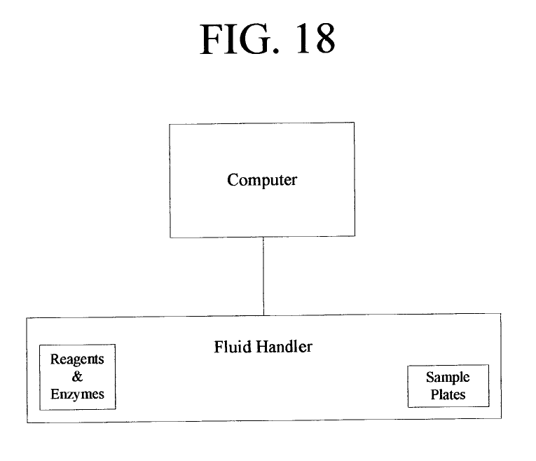

FIG. 18 shows a computer and a TECAN fluid handler for effecting an automated process for practicing the present invention. Specifically, the computer is programmed to control fluid manipulating aspects of the TECAN in order to add the respective sequences, P, I and/or T along with appropriate reagents, enzymes, etc., for effecting the various methods set forth in the drawings and the description above.

Various details of the present invention may be changed without departing from its spirit or its scope. Furthermore, the foregoing description of the embodiments according to the present invention are provided for illustration only, and not for the purpose of limiting the invention as defined by the appended claims and their equivalents.

What is claimed is:

1. A method of joining double stranded polynucleotides which comprises:
a. generating 5′ complementary cohesive ends on two or more polynucleotides by treating the polynucleotides with a DNA polymerase having 3′ to 5′ exonuclease activity and 5′ to 3′ polymerizing activity in the presence of less than all of the dNTPs selected from the group consisting of dGTP, dCTP, dATP and dTTP wherein complementary 5′ overhangs are produced;
b. allowing the complementary cohesive ends to anneal wherein at least one of the polynucleotides has a 5′ phosphate group using a DNA kinase treatment if necessary; and
c. ligating the annealed complementary cohesive ends by treating of a DNA ligase;
wherein the DNA kinase treatment, DNA polymerase treatment, and DNA ligase treatment are performed in any one of the following combinations:
DNA kinase followed by DNA polymerase, followed by DNA ligase;
DNA kinase and DNA polymerase co-treatment followed by DNA ligase treatment;
DNA polymerase, followed by DNA kinase, followed by ligase;
DNA polymerase followed by DNA kinase and DNA ligase co-treatment;
DNA kinase followed by DNA polymerase and DNA ligase co-treatment;
and wherein the DNA kinase treatment may be optional.

2. The method of claim 1, wherein the kinase reaction of at least one of the DNA fragments is stopped prior to being combined with other DNA fragments.

3. The method of claim 1 wherein the 5′ phosphate group is added to the polynucleotide in the presence of the DNA polymerase by adding a polynucleotide kinase to the reaction mixture in step (a).

4. The method of claim 3 wherein the DNA polymerase, DNA ligase and polynucleotide kinase reactions are accomplished concurrently.

5. The method of claim 4 wherein the DNA polymerase and polynucleotide kinase reactions occur before the DNA ligase reaction.

6. The method of claim 4 where the final recombinant molecule(s) will be amplified by PCR.

7. The method of claim 4 where the final recombinant molecule(s) or PCR products thereof are used for in vitro transcription and/or in vitro transcription/translation reactions.

8. The method of claim 4 where the final recombinant molecule(s) or PCR products thereof are used for in vivo transcription and/or in vivo transcription/translation reactions.

9. A method for construction of recombinant DNA molecules as set forth claim 1, or 4, wherein the recombinant DNA is constructed with the use of automated liquid handlers.

10. A method for construction of recombinant DNA molecules through the method of claim 1, or 4 wherein the resulting recombinant DNA is to be replicated in a living cell.

11. A method for construction of recombinant DNA molecules as set forth in claim 1, or 4 where the recombinant DNA is to be used for constructing ds RNAs for applications in RNAi.

12. A method for construction of recombinant DNA molecules as set forth in claim 1, or 4 where the recombinant DNA is to be used for constructing ds hairpin RNAs for applications in RNAi.

13. A method for construction of recombinant DNA molecules as set forth in claim 1, or 4 where the recombinant DNA is to be used in in vitro transcription reactions.

14. A method for construction of recombinant DNA molecules as set forth in claim 1, or 4 where the recombinant DNA is to be used in in vitro transcription reactions.

15. A method for construction of recombinant DNA molecules as set forth in claim 1, or 4 where the recombinant DNA is to be used in in vitro transcription/translation reactions.

16. A method for construction of recombinant DNA molecules as set forth in claim 1, or 4 where the recombinant DNA is to be used in in vivo transcription/translation/gene expression studies.

17. A composition of DNA polymerase, in the presence of a subset of all 4 dNTPs, DNA kinase and DNA ligase activities which function to efficiently generate recombinant DNAs by either maintaining complementary 5′ single stranded overhangs between multiple DNAs, kinasing the 5′ ends of DNAs, if necessary and allowing DNAs to anneal through the complementary overhangs and be covalently joined by the DNA ligase activity.

18. A composition of DNA polymerase, in the presence of a subset of all 4 dNTPs, DNA kinase and DNA ligase activities which function to efficiently generate recombinant DNAs by either generating complementary 5′ single stranded overhangs between multiple DNAs, kinasing the 5′ ends of DNAs, if necessary and allowing DNAs to anneal through the complementary overhangs and be covalently joined by the DNA ligase activity.

19. A composition of DNA polymerase, in the presence of a subset of all 4 dNTPs, and DNA kinase which functions to efficiently generate 5′ single stranded overhangs on at least one end of a ds DNA molecule, and efficiently adds a phosphate group to 5′ ends of DNA, if necessary.

20. A composition of DNA polymerase, in the presence of a subset of all 4 dNTPs, and DNA kinase which functions to efficiently maintain 5′ single stranded overhangs on at least one end of a ds DNA molecule, and efficiently adds a phosphate group to 5′ ends of DNA, if necessary.

21. A composition of DNA polymerase, in the presence of a subset of all 4 dNTPs, and DNA ligase which functions to efficiently generate 5′ single stranded overhangs on at least one end of a ds DNA molecule, and allows DNAs with complementary 5′ single stranded overhangs to anneal and be efficiently joined by DNA ligase.

22. A composition of DNA polymerase, in the presence of a subset of all 4 dNTPs, and DNA ligase which functions to efficiently maintain 5′ single stranded overhangs on at least one end of a ds DNA molecule, and allows DNAs with complementary 5′ single stranded overhangs to anneal and be efficiently joined by DNA ligase.

23. A computer controlled method for effecting any one of the plurality of processes as shown in FIGS. 15-17 for construction of recombinant DNA.

24. An apparatus for computer controlling any one of the plurality of processes as shown in FIGS. 15-17 for construction of recombinant DNA.


SEQUENCE LISTING (52 sequences)

SEQ ID NO: 1 - DNA, 10 bp, Artificial Sequence
  Misc. recombinant sequence
ggttttttgg

SEQ ID NO: 2 - DNA, 10 bp, Artificial Sequence
  Misc. reombinant sequence
ccaaaaaacc

SEQ ID NO: 3 - DNA, 28 bp, Artificial Sequence
  PCR primer
ggccttatggctagcaaaggagaagaac

SEQ ID NO: 4 - DNA, 25 bp, Artificial Sequence
  PCR Primer
gcttgtagagctcatccatgccatg

SEQ ID NO: 5 - DNA, 12 bp, Artificial Sequence
  PCR Primer
gccatgggcatt

SEQ ID NO: 6 - DNA, 32 bp, Artificial Sequence
  PCR Primer
gactcttcaagcgcaagagcgcccaatacgca

SEQ ID NO: 7 - DNA, 29 bp, Artificial Sequence
  PCR Primer
ggcttagctgtttcctgtgtgaaattgtt

SEQ ID NO: 8 - DNA, 238 bp, Artificial Sequence
  Experimental PCR Product
gactcttcaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctaagcc

SEQ ID NO: 9 - DNA, 25 bp, Artificial Sequence
  PCR Primer
gccatggctagcaaaggagaagaac

SEQ ID NO: 10 - DNA, 28 bp, Artificial Sequence
  PCR Primer
cggttatttgtagagctcatccatgcca

SEQ ID NO: 11 - DNA, 726 bp, Artificial Sequence
  Experimental PCR Product
gccatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatgggccttcttgagtttgtaactgctgctgggattacacatggcatggatgagctctacaaataaccg

SEQ ID NO: 12 - DNA, 28 bp, Artificial Sequence
  PCR Primer
ccgttttaagccagccccgaccacccgc

SEQ ID NO: 13 - DNA, 24 bp, Artificial Sequence
  PCR Primer
gatgagcggatacatatttgaatg

SEQ ID NO: 14 - DNA, 302 bp, Artificial Sequence
  Experimental PCR Product
ccgttttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatc

SEQ ID NO: 15 - DNA, 32 bp, Artificial Sequence; identical to SEQ ID NO: 6
  PCR Primer

SEQ ID NO: 16 - DNA, 23 bp, Artificial Sequence
  PCR Primer
aatggctagcaaaggagaagaac

SEQ ID NO: 17 - DNA, 28 bp, Artificial Sequence
  PCR Primer
taaggttatttgtagagctcatccatgc

SEQ ID NO: 18 - DNA, 29 bp, Artificial Sequence
  PCR Primer
attggtagctgtttcctgtgtgaaattgt

SEQ ID NO: 19 - DNA, 29 bp, Artificial Sequence
  PCR Primer
ttaggttaagccagccccgacacccgcca

SEQ ID NO: 20 - DNA, 25 bp, Artificial Sequence
  PCR Primer
ggcatggctagcaaaggagaagaac

SEQ ID NO: 21 - DNA, 28 bp, Artificial Sequence
  PCR Primer
cggttatttgtagagctcatccatgcca

SEQ ID NO: 22 - DNA, 29 bp, Artificial Sequence
  PCR Primer
gccttagctgtttcctgtgtgaaattgtt

SEQ ID NO: 23 - DNA, 29 bp, Artificial Sequence
  PCR Primer
ccgttttaagccagccccgacacccgcca

SEQ ID NO: 24 - DNA, 25 bp, Artificial Sequence
  PCR Primer
aaaatggctagcaaaggagaagaac

SEQ ID NO: 25 - DNA, 29 bp, Artificial Sequence
  PCR Primer
ttaggttatttgtagagctcatccatgcc

SEQ ID NO: 26 - DNA, 29 bp, Artificial Sequence
  PCR Primer
attttcgagctgtttcctgtgtgaaattg

SEQ ID NO: 27 - DNA, 29 bp, Artificial Sequence
  PCR Primer
taaccttaagccagccccgacacccgcca

SEQ ID NO: 28 - DNA, 24 bp, Artificial Sequence
  PCR Primer
ccatggctagcaaaggagaagaac

SEQ ID NO: 29 - DNA, 29 bp, Artificial Sequence
  PCR Primer
gccttatttgtagagctcatccatgccat

SEQ ID NO: 30 - DNA, 28 bp, Artificial Sequence
  PCR Primer
ggttagctgtttcctgtgtgaaattgtt

SEQ ID NO: 31 - DNA, 29 bp, Artificial Sequence
  PCR Primer
ggcttttaagccagccccgacacccgcca

SEQ ID NO: 32 - DNA, 21 bp, Artificial Sequence
  PCR Primer
cggctggtcgacgcgggttta

SEQ ID NO: 33 - DNA, 15 bp, Artificial Sequence
  PCR Primer
aactcaatggtgatg

SEQ ID NO: 34 - DNA, 41 bp, Artificial Sequence
  PCR Primer
aaaaaagctcttctcatcctgacaagaacacgaactgagat

SEQ ID NO: 35 - DNA, 44 bp, Artificial Sequence
  PCR Primer
ttttttgctcttcaaaaccccaagacaattctgcagatatccag

SEQ ID NO: 36 - DNA, 48 bp, Artificial Sequence
  PCR Primer
ttttttgctagcgcggagccgttttttgctcttcaaaaccccaagaca

SEQ ID NO: 37 - DNA, 20 bp, Artificial Sequence
  PCR Product
tgtttagccggtttggtcgt

SEQ ID NO: 38 - DNA, 21 bp, Artificial Sequence
  PCR Primer
ttgatccgttgatcacggcgt

SEQ ID NO: 39 - DNA, 21 bp, Artificial Sequence
  PCR Primer
cactatgcgttatcgtacgca

SEQ ID NO: 40 - DNA, 140 bp, Artificial Sequence
  Experimental PCR Product
tcgttttaaatagatcttacagtatcactactccatctcagttcgtgttcttgtcaggatgagaagagcttttttgctagcgcggagccgttttttgctcttcaaaaccccaagacaattctgcagatatccagcacagt

SEQ ID NO: 41 - DNA, 140 bp, Artificial Sequence
  Experimental PCR Product
agcaaaatttatctagaatgtcatagtgatgaggtagagtcaagcacaagaacagtcctactcttctcgaaaaaacgatcgcgcctcggcaaaaaacgagaagttttggggttctgttaagacgtctataggtcgtgtca

SEQ ID NO: 42 - DNA, 29 bp, Artificial Sequence
  PCR Primer
ggcttagctgtttcctgtgtgaaattgtt

SEQ ID NO: 43 - DNA, 25 bp, Artificial Sequence
  PCR Primer
gccatggctagcaaaggagaagaac

SEQ ID NO: 44 - DNA, 238 bp, Artificial Sequence; identical to SEQ ID NO: 8
  Experimental PCR Product

SEQ ID NO: 45 - DNA, 723 bp, Artificial Sequence
  Experimental PCR Product
gccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatgaccg

SEQ ID NO: 46 - DNA, 30 bp, Artificial Sequence
  PCR Primer
atgcccgctagcaaaggagaagaacttttc

SEQ ID NO: 47 - DNA, 30 bp, Artificial Sequence
  PCR Product
tttccctttgtagagctcatccatgccatg

SEQ ID NO: 48 - DNA, 25 bp, Artificial Sequence
  PCR Primer
cttacgtacttgcaccatccatgga

SEQ ID NO: 49 - DNA, 25 bp, Artificial Sequence
  PCR Primer
cttacgtacttgcaccatccatgga

SEQ ID NO: 50 - DNA, 25 bp, Artificial Sequence
  PCR Primer
aggtacctaccacgttcatgcattc

SEQ ID NO: 51 - DNA, 42 bp, Artificial Sequence
  PCR Primer
cttacgtacttgcaccatccatggatggtgcaagtacgtaag

SEQ ID NO: 52 - DNA, 42 bp, Artificial Sequence
  PCR Primer
gaatgcatgaacgtggtaggtacctaccacgttcatgcattc